SEQUENCE LISTING (9 sequences)

SEQ ID NO: 1 - protein, 28 aa, Homo sapiens
  MOD_RES (3)..(3): Amino acid in position 3 is modified with a fatty acid
GSSFLSPEHQRVQQRKESKKPPAKLQPR

SEQ ID NO: 2 - protein, 27 aa, Homo sapiens
  MOD_RES (3)..(3): Amino acid in position 3 is modified with a fatty acid
GSSFLSPEHQRVQRKESKKPPAKLQPR

SEQ ID NO: 3 - protein, 28 aa, Rattus rattus
  MOD_RES (3)..(3): Amino acid in position 3 is modified with a fatty acid
GSSFLSPEHQKAQQRKESKKPPAKLQPR

SEQ ID NO: 4 - protein, 7 aa, Homo sapiens
FLSPEHQ

SEQ ID NO: 5 - protein, 6 aa, Homo sapiens
FLSPEH

SEQ ID NO: 6 - protein, 5 aa, Homo sapiens
FLSPE

SEQ ID NO: 7 - protein, 4 aa, Homo sapiens
FLSP

SEQ ID NO: 8 - protein, 3 aa, Homo sapiens
FLS

SEQ ID NO: 9 - protein, 2 aa, Artificial
  Synthetic construct
FL

(19) United States
(12) Patent Application Publication
(10) Pub. No.: US 2008/0171700 A1
(43) Pub. Date: Jul. 17, 2008

(54) Use Of Secretagogue For The Teatment Of Ghrelin Deficiency

(75) Inventors: Henrik Nilsson, Copenhagen K (DK); Birgitte Holst Lange, Princeton, NJ (US); Claes Post, Sigtuna (SE); Tina Geritz Nielsen, Frederiksberg C (DK)

Correspondence Address: BROWDY AND NEIMARK, P.L.L.C.;624 NINTH STREET, NW, SUITE 300, WASHINGTON, DC, 20001-5303, US

(73) Assignee: Gastrotech Pharma A/S, Copenhagen K (DK)

(21) Appl. No.: 11/578,135

(22) PCT Filed: Apr. 7, 2005
(86) PCT No.: PCT/DK05/00237

§ 371 Date: Oct. 10, 2007

(30) Foreign Application Priority Data
Apr. 7, 2004 (DK) PA200400569
Oct. 27, 2004 (DK) PA2004016565

(51) Int. Cl. A61K 38/25; A61P 5/00; A61P 3/00; A61P 3/04

(52) U.S. Cl. 514/12

(57) ABSTRACT

The present invention relates to the use of a growth hormone (GH) secretagogue, such as a ghrelin-like compound, for the preparation of a medicament for the prophylaxis or treatment of ghrelin deficiency, and/or undesirable symptoms associated therewith, in an individual at risk of acquiring partial or complete ghrelin deficiency resulting from a medical treatment and/or from a pathological condition. The present invention also relates to use of a secretagogue compound for the preparation of a medicament for the prophylaxis or treatment of one or more of: loss of fat mass, loss of lean body mass, weight loss, cachexia, loss of appetite, immunological dysfunction, malnutrition, disrupted sleep pattern, sleepiness, reduction in intestinal absorption and/or intestinal mobility problems in an individual suffering from, or at risk of suffering from, ghrelin deficiency. Furthermore, the present invention relates to the use of a secretagogue, such as a ghrelin-like compound, for the production of a medicament for preventing weight increase in an individual either: a) being converted from a hyperthyroidic state to euthyroid state, or b) in remission from being converted from a hyperthyroidic state to euthyroid state.

All patent and non-patent references cited in the application, or in the present application, are also hereby incorporated by reference in their entirety.

### FIELD OF INVENTION

The present invention relates to the use of a growth hormone (GH) secretagogue, such as a ghrelin-like compound, for the preparation of a medicament for the prophylaxis or treatment of ghrelin deficiency, and/or undesirable symptoms associated therewith, in an individual at risk of acquiring partial or complete ghrelin deficiency resulting from a medical treatment and/or from a pathological condition. The present invention also relates to use of a secretagogue compound for the preparation of a medicament for the prophylaxis or treatment of one or more of: loss of fat mass, loss of lean body mass, weight loss, cachexia, loss of appetite, immunological dysfunction, malnutrition, disrupted sleep pattern, sleepiness, reduction in intestinal absorption and/or intestinal mobility problems in an individual suffering from, or at risk of suffering from, ghrelin deficiency. Furthermore, the present invention relates to the use of a secretagogue, such as a ghrelin-like compound, for the production of a medicament for preventing weight increase in an individual either:

a) being converted from a hyperthyroidic state to euthyroid state, or
b) in remission from being converted from a hyperthyroidic state to euthyroid state.

The present invention further relates to a method for preventing weight increase in an individual either:

a) being converted from a hyperthyroidic state to euthyroid state, or
b) in remission from being converted from a hyperthyroidic state to euthyroid state; by administering a secretagogue, such as a ghrelin-like compound.

### BACKGROUND OF INVENTION

Ghrelin is a bioactive peptide which originally was described to be involved in the control of GH secretion but later found to be a major regulator of appetite, food intake and energy homeostasis (Kojima M et al., Trends Endocrinol Metab 12:118-122; Nakazato M et al., 2001, Nature 409:194-198). Similar to many other bioactive peptides, ghrelin probably act both as a hormone, a paracrine substance and as a neurotransmitter. The story of ghrelin, its receptor and synthetic compounds acting through this receptor unraveled in a unique “reverse” order. In the eighties a synthetic hexa-peptide from a series of opioid-like peptides was found to be able to release growth hormone (GH) from isolated pituitary cells (Bowers C Y et al., 1980, Endocrinology 106:663-667). Since this action was independent of the growth hormone releasing hormone (GHRH) receptor, several pharmaceutical companies embarked upon drug discovery projects based on this hexa-peptide GH secretagogue (GHS) and its putative receptor. Several series of potent and efficient peptide as well as non-peptide GH secretagogues were consequently described in the mid nineties (Bowers C Y et al., Endocrinology 114:1537-1545; Patchett A A et al., 1995; Proc Natl Acad Sci USA 92:7001-7005; Smith R G et al., Science 260:1640-1643). However, it was only several years later that the receptor through which these artificial GH secretagogues acted was eventually cloned and shown to be a member of the 7™ G protein coupled receptor family (Howard A D et al., Science 273:974-977; Smith R G et al., 1997 Endocr Rev 18:621-645). In 1999, the endogenous ligand for this receptor the hormone ghrelin was finally discovered (Kojima M et al., 1999, Nature 402:656-660). The main site for ghrelin production is the stomach, where the peptide is found in classical endocrine cells in the gastric mucosa.

From here, ghrelin is secreted in the pre-meal situation which results in a sharp, short-lived surge in plasma levels of ghrelin before the meal and starting 1-2 hours before and lasting a short while after initiation of the meal. Since ghrelin is the only peripherally produced orexigenic (appetite promoting) substance it is believed that the increase in plasma levels of ghrelin is crucial for the initiation of the meal.

In its role as a key initiator of appetite, ghrelin released from the endocrine cells in the mucosa of the GI tract may act both locally as a paracrine substance and centrally as a hormone.

### Ghrelin Deficiency

An individual with ghrelin deficiency lacks sufficient levels of the peptide hormone ghrelin. Ghrelin deficiency is associated with a number of pathological causes, however until now was not deemed in itself to be a significant cause of further pathology. Indeed, a ghrelin-deficient mouse has been generated that showed that ghrelin is not a vital regulator of mouse bodily systems: the deficient mice had the same size, growth rate, food intake, body composition, reproduction, gross behaviours and tissue pathology as their healthy littermates Sun et al., Molecular and Cellular Biology, 23 (22): 7973-7981, “Deletion of Ghrelin affects neither growth nor appetite”). Thus, ghrelin deficiency could be considered an effect of pathology rather than a cause of further pathology.

Although ghrelin deficiency is a known phenomenom in some cases, e.g. hyperthyroidism, it was previously thought that ghrelin deficiency would lead to compensatory upregulation of GHS-1a receptor expression, which would then induce increased sensitivity to the hormone. Certainly, no distinct ghrelin deficiency syndrome has been documented until now, nor was a need to administer ghrelin to ghrelin-deficient patients documented.

### SUMMARY OF INVENTION

The present invention relates to use of a secretagogue compound, such as ghrelin or a ghrelin-like compound, for the preparation of a medicament for the prophylaxis or treatment of ghrelin deficiency, and/or symptoms associated with ghrelin deficiency, in an individual in need thereof. Said individual may be suffering from, or at risk of acquiring, partial or complete ghrelin deficiency resulting from e.g. a medical treatment or pathological condition. In all embodiments described herein, it is also envisaged that a secretagogue, such as a ghrelin-like compound, may be used to treat/prevent symptoms in those who have previously suffered, or are in remission from, ghrelin deficiency.

It is surprising and unexpected that the effect of ghrelin deficiency on humans is very different than in a knockout mouse model. The ghrelin deficiency syndrome in the human being has been found by the inventors of the present invention to be associated with one or more of the following symptoms: loss of fat mass, loss of lean body mass, weight loss, cachexia, loss of appetite, immunological-disruption and malnutrition, disrupted sleep pattern, sleepiness, malabsorption and motility problems with the intestine. Never before has it been realised that ghrelin deficiency has such side-effects in the human patient.

The present invention also relates to use of a secretagogue compound for the preparation of a medicament for the prophylaxis or treatment of one or more of: loss of fat mass, loss of lean body mass, weight loss, cachexia, loss of appetite, immunological dysfunction, malnutrition, disrupted sleep pattern, drowsiness, lowered intestinal absorption and/or intestinal motility problems in an individual suffering from, or at risk or suffering from, a pathological condition selected from:
a pathological condition associated with insulin resistance
a pathological condition associated with disrupted epithelium in the GI tract
hyperthyroidism

The present invention also relates to a method of treatment of an individual suffering from ghrelin deficiency, wherein said individual is administered a GH secretagogue compound or pharmaceutical salt thereof. The present invention further relates to a method of treatment of one or more of: loss of fat mass, loss of lean body mass, weight loss, cachexia, loss of appetite, immunological dysfunction, bone remodulation, malnutrition, disrupted sleep pattern, drowsiness, lowered intestinal absorption and/or intestinal motility problems in an individual suffering from, or at risk or suffering from, a pathological condition selected from:
a pathological condition associated with insulin resistance
a pathological condition associated with disrupted epithelium in the GI tract
hyperthyroidism.

It is preferred that administration of the compounds of the present invention acts to prevent or reverse a ghrelin-deficient state of an individual.

In another aspect, the invention relates to the use of a secretagogue compound for the preparation of a medicament for preventing weight increase in individuals being converted from a hyperthyroidic state to euthyroid state and/or in remission from a state of hyperthyroidism. It is preferred that said individual is being converted from a hyperthyroidic state to euthyroid state Furthermore, the invention relates to a method for preventing weight increase in individuals being converted from a hyperthyroidic state to euthyroid state and/or in remission from a state of hyperthyroidism, said method comprising administration of a secretagogue to said individual.

Preferably, the secretagogue used in the uses and methods of the present invention is a ghrelin-like compound which comprises a structure defined herein below, or a pharmaceutically acceptable salt thereof.

In order to minimize weight gain, secretagogue therapy is preferably initiated at the time of referral to treatment of hyperthyroidism and continued throughout the treatment period (and optionally after, during the remission period), in doses that at least normalize the individual's plasma ghrelin level, thus preventing an upregulation in the number of the individual's ghrelin receptors

In a preferred aspect of the invention the secretagogue, such as a ghrelin-like compound is administered with a substance capable of increasing the half-life of the secretagogue, for example by incorporating the secretagogue compound into liposomes, micelles, iscoms, and/or microspheres or other transport molecules, in particular to protect the modified amino acid from being desacylated.

In all embodiments of the present invention, the medicament can be administered as a bolus injection or by fast running infusion, i.e. an infusion preferably lasting less than 120 minutes, such as less than 90 minutes, for example less than 60 minutes, such as less than 45 minutes, such as less than 30 minutes, for example less than 25 minutes, such as less than 20 minutes, such as less than 15 minutes, for example less than 12 minutes, such as less than 10 minutes, such as less than 8 minutes, for example less than 6 minutes, such as less than 5 minutes, such as less than 4 minutes, for example less than 3 minutes, such as less than 2 minutes, such as less than 1 minute.

In one preferred embodiment the medicament is administered as a bolus. The bolus is preferably administered subcutaneously.

Prevention of Weight Gain in Individuals being Converted from a Hyperthyroidic State to Euthyroid State.

Without being bound by theory, the low plasma ghrelin level and the general state of starvation in hyperthyroidism may induce an increase in ghrelin receptor level in the hypothalamus. This up regulation may last much longer than the actual increase in thyroid function—but due to the constitutive activity of the ghrelin receptor the hypothalamus will respond with increased food intake. Treatment of an individual with such an altered metabolism with a secretagogue will prevent upregulation of the ghrelin receptors, thus preventing or lessening high levels of food intake, and decreasing or preventing weight gain.

Administration of a secretagogue such as ghrelin may also act to prevent or lessen the increase in body weight observed the following 1-5 years after euthyroid conditions have been achieved by e.g. anti-thyroid treatment or radioiodine treatment. Thus, ghrelin can also prevent increase in body weight during remission from hyperthyroid treatment, when the individual is still at risk of weight increase due to altered metabolism, such as the following 1-5 years after euthyroid conditions have been achieved.

These effects probably also work in synergy with the contributing effect that ghrelin decreases locomotor activity which may be helpful in order to relax the restless patients with hyperthyroid diseases. Another contributing effect may also be that preventing a weight gain or facilitating maintenance of weight, in particular in individuals being converted from a hyperthyroidic state to euthyroidic state, is correcting the imbalance between energy intake and energy consumption, i.e. total body metabolism. During the hyperthyroid period a secretagogue such as ghrelin may counteract the increased metabolism and hence increase body weight, decrease body temperature and minimize the catabolic condition. In addition ghrelin has also been shown to decrease locomotor activity which may be helpful in order to relax the restless patients with hyperthyroid diseases.

### DETAILED DESCRIPTION OF THE INVENTION

### Definitions

Amino acid: Entity comprising an amino terminal part (NH₂) and a carboxy terminal part (COOH) separated by a central part comprising a carbon atom, or a chain of carbon atoms, comprising at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822(b)(2) belong to the group of amino acids listed in Table 1 herein below. Non-natural amino acids are those not listed in Table 1. Examples of non-natural amino acids are those listed e.g. in 37 C.F.R. section 1.822(b)(4), all of which are incorporated herein by reference. Further examples of non-natural amino acids are listed herein below. Amino acid residues described herein can be in the “D” or “L” isomeric form.

TABLE 1
Natural amino acids and their respective codes.

Symbols

| | | |
|---|---|---|
| 1-Letter | 3-Letter | Amino acid |

| | | |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

Appetite: Appetite in an individual is assessed by measuring the amount of food ingested and by assessing the individual's desire to eat. Appetite (i.e., hunger) is typically assessed with a short questionnaire given to individuals on a random basis several times a week. Typically, subjects rate their hunger, preoccupation with food, and desire to eat greater quantities and different types of food by answering the questions using analogue scales ranging from 1, not at all, to 5, extremely.

Amino acid residue: the term “amino acid residue” is meant to encompass amino acids, either standard amino acids, non-standard amino acids or pseudo-amino acids, which have been reacted with at least one other species, such as 2, for example 3, such as more than 3 other species. In particular amino acid residues may comprise an acyl bond in place of a free carboxyl group and/or an amine-bond and/or amide bond in place of a free amine group. Furthermore, reacted amino acids residues may comprise an ester or thioester bond in place of an amide bond

BMI: The body mass index (BMI) measures an individual's height to weight ratio. It is determined by calculating weight in kilograms divided by the square of height in meters. The BMI “normal” range is 19-22.

Body fat mass: Body fat mass can be measured e.g. by the fat fold technique: In this technique, a pincer-type caliper is used to measure subcutaneous fat by determining skin fold thickness at representative sites on the body. These skin fold measurements are then used to compute body fat by either adding the scores from the various measurements and using this value as an indication of the relative degree of fatness among individuals or by using the measurements in mathematical equations that have been developed to predict percent body fat. Another measuring method that can be used to calculate body fat mass is a DEXA scan.

Cachexia: a wasting disorder, the symptoms of which comprise weight loss, wasting of muscle, loss of appetite, and general debilitation. These symptoms are often associated with chemotherapeutic treatment regimes.

Chemotherapy: herein, the term “chemotherapy” refers to any treatment of an individual with a cytotoxic drug, usually causing a reduction in bone marrow content. By “cytotoxic drug” is meant a drug that kills or arrests the growth of cells, preferably by targeting specific parts of the cell growth cycle. Diseases that may be treated by chemotherapy include metastatic cancers.

Concentration equivalent: A concentration equivalent is an Equivalents dosage being defined as the dosage of a secretagogue having in vitro and/or in vivo the same response as evaluated from a dosage-response curve as wild-type ghrelin.

Ghrelin: a polypeptide as described in Kojima M, Hosoda H, Date Y, Nakazato M, Matsuo H, Kangawa K 1999 Ghrelin is a growth-hormone-releasing acylated peptide from stomach. Nature 402:656-660. Human 28 aa ghrelin has the amino acid of SEQ ID NO: 1.

Ghrelin analogues: The present invention also embraces the use of ghrelin analogues. In the context of the present application, analogues to ghrelin are to be understood as any peptide or non-peptide compound that essentially exerts the same biological effect as ghrelin in vivo. Exemplary non-peptide ghrelin analogues are described in EP 0 869 974 and EP 1 060 190, which illustrate a number of ghrelin analogues and which documents are incorporated herein by way of reference. Any of the analogues mentioned in the documents referred to herein may be utilized. Preferred compounds are the compounds designated as NN 703 [5-Amino-5-methylex-2-enoic acid N-methyl-N-((1R)-1-(methyl-((1R)-1-(methylcarbamoyl-2-phenylethylcarbomoyl)-2-(naphtalen-2-yl)ethyl)amide] and MK677 [sometimes also designated MKO677, cf. Drug Discovery Today, vol. 4, No. 11, November 1999, 497-506] or NNC 26-1291, or NNC 26-1187 are growth hormone secretagogues of a non-peptidyl described in WO 99/58501 and WO 00/26252, respectively, all of which documents are incorporated herein by way of reference.

Ghrelin-like compound: the term “ghrelin-like compound” as used herein refers to any compound which mimics the function of wild-type ghrelin, in particular wild-type human ghrelin, particularly in terms of the ghrelin functions leading to the desired therapeutic effects described herein, and is preferably defined by the formula I:

Z¹-(X¹)ₘ—(X²)—(X³)ₙ-Z², wherein

Z¹ is an optionally present protecting group
each X¹ is independently selected from an amino acid, wherein said amino acid is selected from naturally occurring and synthetic amino acids,
X² is any amino acid selected from naturally occurring and synthetic occurring amino acids, said amino acid being modified with a bulky hydrophobic group, preferably an acyl group, or a fatty acid,
each X³ is independently selected from an amino acid, wherein said amino acid is selected from naturally occurring and synthetic amino acids,
wherein one or more of X¹ and X³ optionally may be modified by a bulky hydrophobic group, preferably an acyl group, or a fatty acid,
Z² is an optionally present protecting group,
m is an integer in the range of from 1-10
n is 0 or an integer in the range of from 1-35.

Ghrelin deficiency: There are a number of methods for measuring ghrelin deficiency, and the “levels” of ghrelin calculated using these methods are not always directly comparable. For the purposes of this disclosure, “ghrelin deficiency” is defined using one of the following methods, or an equivalent method within the skill of one skilled in the art:
(a) The method of Marchesini et al., J. Clin. Endocrinol. Metab, 2003 December; 88(12): 5674-9
   this method calculates normal fasting ghrelin levels as 401 fmol/ml with a range of error of 130 fmol/ml. Using this assay method, it is to be understood herein that ghrelin deficiency is diagnosed when an individual is measured as having a fasting ghrelin level lower than 265 fmol/ml, such as lower than 255 fmol/ml, such as lower than 245 fmol/ml, such as lower than 235 fmol/ml, such as lower than 225 fmol/ml, such as lower than 215 fmol/ml, such as lower than 205 fmol/ml, such as lower than 195 fmol/ml, such as lower than 185 fmol/ml, such as lower than 175 fmol/ml, such as lower than 165 fmol/ml, such as lower than 155 fmol/ml, such as lower than 145 fmol/ml.
(b) The method of Ariyasu et al., Endocrinology 2002, 143(9):3341-3351
   this method calculates normal fasting ghrelin levels as 150 fmol/ml with a range of error of 40 fmol/ml. Using this assay method, it is to be understood herein that ghrelin deficiency is diagnosed when an individual is measured as having a fasting ghrelin level lower than 105 fmol/ml, such as lower than 100 fmol/ml, such as lower than 95 fmol/ml, such as lower than 90 fmol/ml, such as lower than 85 fmol/ml, such as lower than 80 fmol/ml, such as lower than 75 fmol/ml, such as lower than 70 fmol/ml, such as lower than 65 fmol/ml, such as lower than 60 fmol/ml.
(c) The method of Enomoto et al., Clinical Science 105, 431-435, 2003
   this method calculates normal fasting ghrelin levels as 150 fmol/ml. Using this assay method, it is to be understood herein that ghrelin deficiency is diagnosed when an individual is measured as having a fasting ghrelin level lower than 130 fmol/ml, such as lower than 125 fmol/ml, such as lower than 120 fmol/ml, such as lower than 115 fmol/ml, such as lower than 110 fmol/ml, such as lower than 105 fmol/ml, such as lower than 100 fmol/ml, such as lower than 95 fmol/ml, such as lower than 90 fmol/ml, such as lower than 85 fmol/ml, such as lower than 80 fmol/ml, such as lower than 75 fmol/ml, such as lower than 70 fmol/ml, such as lower than 65 fmol/ml, such as lower than 60 fmol/ml.
(d) The method of Cummings et al., New England Journal of Medicine, 2002, 346(21):1623-30
   this method calculates normal ghrelin levels as 192 fmol/ml at breakfast peak. Using this assay method, it is to be understood herein that ghrelin deficiency is diagnosed when an individual is measured as having a ghrelin level lower than 175 fmol/ml at breakfast peak, such as lower than 170 fmol/ml, such as lower than 165 fmol/ml, such as lower than 160 fmol/ml, such as lower than 155 fmol/ml, such as lower than 150 fmol/ml, such as lower than 145 fmol/ml, such as lower than 140 fmol/ml, such as lower than 135 fmol/ml, such as lower than 130 fmol/ml, such as lower than 125 fmol/ml, such as lower than 120 fmol/ml, such as lower than 115 fmol/ml, such as lower than 110 fmol/ml, such as lower than 105 fmol/ml
(e) The method of Arioso et al., J. Clin. Endocrinol Metab; 2003, 88(2):701-4
   this method calculates normal fasting ghrelin levels as 1967 fmol/ml. Using this assay method, it is to be understood herein that ghrelin deficiency is diagnosed when an individual is measured as having a fasting ghrelin level lower than 1800 fmol/ml, such as lower than 1700 fmol/ml, such as lower than 1600 fmol/ml, such as lower than 1500 fmol/ml, such as lower than 1400 fmol/ml, such as lower than 1300 fmol/ml, such as lower than 1200 fmol/ml, such as lower than 1100 fmol/ml, such as lower than 1000 fmol/ml, such as lower than 900 fmol/ml, such as lower than 800 fmol/ml, such as lower than 700 fmol/ml, such as lower than 600 fmol/ml, such as lower than 500 fmol/ml, such as lower than 400 fmol/ml.
(f) The method of Stoeckli et al., 2004, 12(2):346-50—this method calculates normal fasting ghrelin levels as 553 pg/ml, (164 fmol/mL) with a range of error of 105 pg/mL. Using this assay method, it is to be understood herein that ghrelin deficiency is diagnosed when an individual is measured as having a fasting ghrelin level lower than 400 pg/ml, such as lower than 380 pg/ml, such as lower than 360 pg/ml, such as lower than 340 pg/ml, such as lower than 320 pg/ml, such as lower than 300 pg/ml, such as lower than 280 pg/ml, such as lower than 260 pg/ml, such as lower than 240 pg/ml, such as lower than 220 pg/ml, such as lower than 200 pg/ml, such as lower than 180 pg/ml, such as lower than 160 pg/ml, such as lower than 140 pg/ml, such as lower than 120 pg/ml.

It is most preferred for the purposes of the present invention that ghrelin deficiency is defined using the method of Cummings, Enomoto or Ariasu, most preferably the method of Cummings

Other indicators associated with ghrelin deficiency may also be taken into account when assessing ghrelin deficiency, such as lowered HDL cholesterol and increased insulin resistance, both correlated with ghrelin deficiency. It is also herein envisaged that one skilled in the art will also take other factors such as an individual's age, sex and physical size into consideration when making a diagnosis of ghrelin deficiency.

GHS: growth hormone secretagogue, also referred to herein as “secretagogue” or “GH secretagogue”.

GHS-R 1a: the receptor for GHS. GHS-R 1a is also denoted GHS 1a. The receptor has GENBANK accession number NM_{—}198407

Immunological dysfunction: by “immunological dysfunction” and grammatical variants thereof is meant any disorder of the immune system, such as immunosuppression, increased activity of the immune system and autoimmune disorders. An example of a disorder of the immune system is an autoimmune disease, such as Grave's disease.

Individual: A living animal or human. In preferred embodiments, the subject is a mammal, including humans and non-human mammals such as dogs, cats, pigs, cows, sheep, goats, horses, rats, and mice. In the most preferred embodiment, the subject is a human.

Isolated: is used to describe any of the various secretagogues, polypeptides and nucleotides disclosed herein, that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified.

“Loss of body weight”: defined herein as a reduction in BMI.

“Loss of body fat”: defined herein as either a reduction of an individual's overall fat mass or a reduction in the percentage of an individual's body fat.

Medical treatment: The term ‘medical treatment’ as used herein refers to any food, drug, device, or procedure that is used and intended as a cure, mitigation, treatment, or prevention of disease and/or a pathological condition.

Modified amino acid: an amino acid wherein an arbitrary group thereof is chemically modified. In particular, a modified amino acid chemically modified at the alpha-carbon atom in an alpha-amino acid is preferable.

Monoclonal Antibody: The phrase monoclonal antibody in its various grammatical forms refers to a population of antibody molecules that contains only one species of antibody combining site capable of immunoreacting with a particular antigen.

Non-acylated ghrelin-like compound: a ghrelin like-compound as defined herein, which does not contain an acyl group attached to any of its constitutent amino acids.

Palliative treatment: a treatment which relieves or soothes the symptoms of a disease or disorder but without effecting a cure.

Polyclonal antibody: Polyclonal antibodies are a mixture of antibody molecules recognising a specific given antigen, hence polyclonal antibodies may recognise different epitopes within said antigen.

Polypeptide: The phrase polypeptide refers to a molecule comprising amino acid residues which do not contain linkages other than amide linkages between adjacent amino acid residues.

Pathological condition: by “pathological condition” is meant any disease or syndrome having a detrimental effect on an individual's physical and/or mental health. Said pathological condition may have a genetic cause. Preferably, the pathological condition treated using the compounds of the present invention leads to one or more undesirable symptoms including loss of fat mass, loss of lean body mass, weight loss, cachexia, loss of appetite, immunological dysfunction, Bone remodulation, malnutrition, disrupted sleep pattern, drowsiness, lowered intestinal absorption and/or intestinal motility.

Peptide: Plurality of covalently linked amino acid residues defining a sequence and linked by amide bonds. The term is used analogously with oligopeptide and polypeptide. The amino acids may be both natural amino acids and non-natural amino acids, including any combination thereof. The natural and/or non-natural amino acids may be linked by peptide bonds or by non-peptide bonds. The term peptide also embraces post-translational modifications introduced by chemical or enzyme-catalyzed reactions, as are known in the art. Such post-translational modifications can be introduced prior to partitioning, if desired. Amino acids as specified herein will preferentially be in the L-stereoisomeric form. Amino acid analogs can be employed instead of the 20 naturally-occurring amino acids. Several such analogs are known, including fluorophenylalanine, norleucine, azetidine-2-carboxylic acid, S-aminoethyl cysteine, 4-methyl tryptophan and the like.

Furthermore, it should be noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

Receptor: A receptor is a molecule, such as a protein, glycoprotein and the like, that can specifically (non-randomly) bind to another molecule.

Remission: an individual is “in remission” from a pathological condition if they are still suffering from (to any extent), or at risk of suffering from, either the symptoms or consequences of the pathological condition they suffered and/or from the effects of the treatment itself (in particular, side effects of the treatment they received). Herein, it is particularly desired that an individual “in remission” from being converted from a hyperthyroidic state to euthyroid state has a greater risk of weight gain than the average healthy individual of the same age.

Secretagogue: a growth hormone secretagogue, i.e. a substance stimulating growth hormone release, such as ghrelin or a ghrelin-like compound. A secretagogue according to the invention may for example be selected from the group of:

L-692-429, L-692-585 (Benzoelactam compounds)

### MK677 (Spiroindaner)

G-7203, G-7039, G-7502 (Isonipecotic acid peptidomimetic)
NN₇O₃, ipamorelin.

In particular the secretagogue is a ghrelin-like compound, including 28 aa human ghrelin. The secretagogue may in one embodiment be non-acylated, for instance a non-acylated form of ghrelin or a non-acylated ghrelin-like compound.

Sequence homology: In one embodiment, sequence homology refers to a comparison made between two molecules using standard algorithms well known in the art. The preferred algorithm for calculating sequence homology for the present invention is the Smith-Waterman algorithm, where e.g. SEQ ID NO:1 is used as the reference sequence to define the percentage identity of polypeptide homologs over its length. The choice of parameter values for matches, mismatches, and inserts or deletions is arbitrary, although some parameter values have been found to yield more biologically realistic results than others. One preferred set of parameter values for the Smith-Waterman algorithm is set forth in the “maximum similarity segments” approach, which uses values of 1 for a matched residue and −⅓ for a mismatched residue (a residue being either a single nucleotide or single amino acid) (Waterman, Bull. Math. Biol. 46, 473-500 (1984)). Insertions and deletions (indels), x, are weighted as

xk=1+k/3,

where k is the number of residues in a given insert or deletion (Id.).

Surfactant molecule: Molecule comprising a hydrophobic part and a hydrophilic part, i.e. molecule capable of being present in the interphase between a lipophilic phase and a hydrophilic phase.

### DETAILED DESCRIPTION OF THE INVENTION

The present invention relates to use of a secretagogue compound, such as a ghrelin-like compound, for the preparation of a medicament for the prophylaxis or treatment of ghrelin deficiency and/or symptoms associated with ghrelin deficiency, in an individual in need thereof. Preferably, said undesirable symptoms include one or more of: loss of fat mass, loss of lean body mass, weight loss, cachexia, loss of appetite, immunological dysfunction, bone malnutrition, disrupted sleep pattern, sleepiness, reduction in intestinal absorption and/or intestinal motility problems.

In particular the present invention relates to treatment and/or prevention of loss of body weight, lean body mass and body fat, or stimulation of weight gain, more preferably treatment and/or prevention of loss of body weight, lean body mass and body fat. Treatment and prevention is seen when an already arising weight loss is stopped from progressing and/or weight gain is initiated. This is probably due to the effect of ghrelin or its analogues to stimulate appetite, and thereby stimulate of food intake, and also ghrelin's effect on an individual's metabolism and body composition. The present invention also relates to stimulation of appetite and stimulation of food intake, more specifically to stimulation of appetite, in individuals at risk of acquiring partial or complete ghrelin deficiency. In another embodiment, it is envisaged that a secretagogue such as ghrelin may be used as a substance to increase the anabolic factor IGF-1, and that as a result leads to increased body weight and/or prevention of loss of body weight and body fat. In one preferred embodiment, the present invention relates to increasing lean body mass and/or prevention of loss of lean body mass.

### Causes of Ghrelin Deficiency

It is envisaged that the methods of the present invention may be used to treat any undesirable side effects of ghrelin deficiency, such as one or more of: loss of fat mass, loss of lean body mass, weight loss, cachexia, loss of appetite, immunological dysfunction and malnutrition, disrupted sleep pattern, sleepiness, malaborption and motility problems with the intestine. Some specific examples of medical treatments and syndromes associated with ghrelin deficiency which may be treated by the methods of the present invention are described below:

### (i) Insulin Resistance Syndromes

Low plasma ghrelin levels are observed in several pathological conditions characterized by insulin resistance, such as polycystitic ovary syndrome, acromegaly and primary/secondary hypogonadism. Many of these conditions are associated with obesity, which is well known to be correlated with low level of plasma ghrelin. However, a low level of plasma ghrelin has also been observed in conditions with insulin resistance but normal or low BMI.

Two examples of this are mentioned below:
Non-Alcoholic Fatty Liver Disease (NAFLD) is significantly associated with metabolic syndrome. Although most patients are overweight or obese 10-20% of the patients have a BMI within normal limits. Insulin resistance measured by homeostasis model assessment is found in almost all patients suffering from NAFLD, including those with normal BMI. It has been shown that NAFLD patients independent of BMI have a low level of plasma ghrelin (see e.g. Marchesini G et al., Low ghrelin concentrations in nonalcoholic fatty liver disease are related to insulin resistance. J Clin Endocrinol Metab. 2003 December; 88(12):5674-9).
Type I Diabetes Mellitus: In pediatric research it has been observed that patients with newly diagnosed Type I DM are dys-regulated in terms of many different hormone levels. The observed low level of Leptin, IGFBP-3 and IGF-I are normalized as the patient are treated with insulin, however significantly low level of ghrelin is not normalized even 4 month after the treatment are initialized.

Treatment of individuals suffering from insulin resistance syndromes with ghrelin or an analogue thereof is new and surprising, as the acylated form of ghrelin is in fact known to be diabetogenic in mouse models (Clark et al., Endocrinology, Vol. 138, no 10., p 4316-4323), therefore there would be no obvious benefit in administering ghrelin or an analogue thereof to a patient. The inventors of the present invention have however found that the benefits accrued by administering ghrelin to prevent the hitherto unknown symptoms of human ghrelin deficiency outweigh the risks of administration, due to an advantageous equilibrium between triglycerides accumulated in the liver compared to the muscles. Preferably, the insulin resistance syndrome treated using the present invention is hyperinsulinemia.

### (ii) Disruption of the Epithelium in the GI Tract:

The inventors of the present invention have found that lowered ghrelin levels are associated with disruption of the epithelium in the GI tract. Without being bound by theory, it is hypothesised that this effect is due to the effects of the disruption affect endocrine cells of the epithelium, in which case a series of important hormones may be suppressed. Most of these hormones are involved with digestion of the meal, however other hormones like ghrelin produced in the stomach may be more important for appetite and body composition.

Different pathological conditions as well as some medical treatments may induce disruption of the epithelium in the gastrointestinal (GI) tract. One cause of disruption of the epithelium in the GI tract is chemotherapy. Chemotherapy is an established technique in the treatment of neoplastic conditions of various types, and acts by targeting cytotoxic agents to cells which grow and multiply rapidly. Side effects related to chemotherapy can be related to the unavoidable non-selective damage of normal and rapidly regenerating cells, which involves structures, such as hair follicle cells, bone marrow, sperms and ova, and the epithelium lining the mouth and the entire GI tract. Damage to the gut lining may also cause nausea and diarrhea, two factors which contribute to side effects of chemotherapy including loss of fat mass, loss of lean body mass, weight loss, cachexia, immunological dysfunction and malnutrition. Chemotherapy may also trigger a loss of appetite, which is also a contributory factor leading to loss of lean body mass, fat mass, weight loss, cachexia, immunological dysfunction and malnutrition. There are thus many undesirable side-effects caused by current chemotherapeutic techniques. The present invention provides a medicament for prophylaxis or treatment of the side-effects caused by disruption of the epithelium of the GI tract by administration of a GH secretagogue, such as ghrelin or an analogue thereof.

Other causes of disruption to the epithelial tract, which may lead to ghrelin deficiency, include radiotherapy and gastristis (causing e.g. atrophy of epithelia in the stomach and damaging endocrine cells), which may be caused by a variety of pathological factors. Individuals suffering from ghrelin deficiency caused by disruption of the GI tract, preferably due to the causes described above, will benefit from administration of a secretagogue compound, such as ghrelin or a ghrelin-like compound. This is surprising, as it was hitherto unknown that the disruption of the GI tract causes ghrelin deficiency, and it was also not known that ghrelin deficiency causes pathological effects.

(iii) Hyperthyroidism

Hyperthyroidism is common affecting approximately 2-5% of all females at some time and with sex ratio of 5:1 most common between 20 and 40 years. Nearly all cases are caused by intrinsic thyroid diseases and only a very few cases are by pituritary disorders.

Hyperthyroidism may be caused by a number of different factors, such as Grave's disease, drugs containing a high level of iodine, thyroiditis (such as subacute thyroiditis and postpartum thyroiditis), loss of feedback control of thyroid hormone producing cells, solitary adenoma, De Quervain thyroiditis, toxic nodular goiter or excessive doses of thyroid hormone, for instance in the case of patients who take forms of thyroid medication that contains T3.

Graves' disease is the most common cause of hyperthyroidism and is caused by autoimmune processes. Serum IgG antibodies acts like the endogenous thyroid stimulating hormone (TSH) and binds to the thyroid binds the thyroid THS receptor stimulating thyroid hormone production. Toxic solitary adenoma, toxic multinodular goiter and De Quervain thyroiditis constitute approximately 5-10% of the total number of hyperthyroid diseases.

The clinical features of hyperthyroid are following:
Due to increased metabolism: weight loss, increased appetite, restlessness, malaise, muscle weakness, tremor, breathlessness and heat intolerance.
Most likely caused by indirect effect on the sexual system:
oligomenorhea, loss libido and gynaecomastia.
Cardiac effects: palpitation due to tachycardia or atrial fribrillation, systolic hypertension and cardia failure.
Due to increased gastric motility: vomiting and diarrhea (only observed in a minor subpopulation of patients where auto-antibodies cross reacts with receptors in the GI-tract.
Eye symptoms are only observed associated with graves disease.
Behavioral changes including irritability, disrupted sleeping and psychosis.

Diagnosis is based on the clinically observed symptoms and on suppressed TSH (<0.1 mU/Litre) and is confirmed by a rise in T3 and T4.

Current treatment: Three different possibilities are available: antithyroid drugs, surgery and radioiodine—in combination with drugs targeting the cardiovascular system. Practices differ widely with in and between countries but large goiters and multinodular and single nodular goiters are not very responsive to anti thyroid treatment.

Both patients treated with thioamides and radioiodine treatment show an increase in body weight in the following 2-5 years after normalization of the thyroid parameters. The effect is strongest after surgery and in patients that have transient hypothyroid periods, however even in patients without hypothyroid function become obese. The weight gain is approximately 3-5 kg pr year. Being overweight or obese is in itself a major cause of further health problems, therefore there is a need for treatments that prevent excessive weight gain associated with hyperthyroid treatment.

Hyperthyroidism is associated with suppressed circulating ghrelin levels (Riis A L, et al., Hyperthyroidism is associated with suppressed circulating ghrelin levels, J Clin Endocrinol Metab. 2003 February; 88(2):853-7). The reason for this increase in plasma ghrelin is not understood but since a concomitant high level of leptin is observed the situation may be compared to the situation observed in patients with low adipose tissue capacity—obese and lipodystrophic patients.

Ghrelin treatment in the very early phase of hyperthyroidism may have two different purposes:
1) Decrease the metabolic rate and increase the capacity of the adipose tissue. The ghrelin induced decrease in cytokine IL-1 (α and β), IL-6 and TNFα may in case of the autoimmune Graves diseases also contribute to a less pronounced development of the hyperthyroid symptoms.
2) Prevent an compensatory increase in the GHS-R1a expression in the hypothalamic area, that may contribute to the obesity and increase in appetite observed 2-5 years following hyperthyroid treatment.

### (iv) Other Ghrelin Deficiency Cases

Some causes of ghrelin deficiency may be due to mutations in the ghrelin gene, which lead to lowered levels and/or lowered levels of active circulating ghrelin. For example, the ghrelin Arg51Gln mutation is associated with low plasma ghrelin concentrations (Poykko et al., “Low plasma ghrelin is associated with insulin resistance, hypertension, and the prevalence of type 2 diabetes” Diabetes. 2003 October; 52(10):2546-53”).

Thus, in one preferred embodiment of the present invention, the individual treated is at risk of acquiring, or has acquired, partial or complete ghrelin deficiency resulting from a pathological condition. In one preferred embodiment, said pathological condition is associated with insulin resistance. Preferably, said condition associated with insulin resistance is selected from the group consisting of: Non-Alcoholic Fatty Liver Disease (NAFLD) and/or Type I Diabetes Mellitus. In another preferred embodiment, said condition is selected from the group consisting of polycystitic ovary syndrome, acromegaly and primary/secondary hypogonadism. In another preferred embodiment, said pathological condition is hyperthyroidism. Preferably, said hyperthyroidism is caused by one or more of the following: Grave's disease, drugs containing a high level of iodine, thyroiditis, subacute thyroiditis, postpartum thyroiditis, loss of feedback control of thyroid hormone producing cells, toxic nodular goiter, excessive doses of thyroid hormone or thyroid medication.

In another preferred embodiment of the present invention, the individual in need of treatment with the compounds of the present invention is suffering from, or at risk of suffering from, ghrelin deficiency associated with disrupted epithelium in the GI tract. By “disrupted epithelium” is meant herein that at least part of the epithelium is damaged, i.e. the damage does not necessarily have to affect the entirety of the epithelium. Said disruption of the epithelium is preferably caused by one or more of: a pathological condition, a genetic disease, or a medical treatment. It is envisaged that the compounds of the present invention may be administered to a patient that has been, will be, or is currently, treated using said medical treatment. Said medical treatment is preferably chemotherapy. In another preferred embodiment, said medical treatment is radiotherapy, which may be used in combination with chemotherapy treatment. In another preferred embodiment of the present invention, said disruption of epithelium in the GI tract is caused by gastritis.

In another preferred embodiment of the present invention, said individual in need of treatment with the compounds of the present invention has a genetic mutation associated with low plasma ghrelin concentrations, such as the Arg51Gln ghrelin mutation.

It is preferred that the individual in need of treatment with the compounds and methods of the present invention has not undergone a gastrectomy, i.e. said individual has not undergone (e.g.) a surgical procedure to remove at least part of said individual's stomach, for example said individual has a anatomically intact stomach. Thus, it is preferred that said individual is not gastrectomized.

In one preferred embodiment of the present invention, the individual in need of treatment does not have an abnormally low number of ghrelin-producing cells, but is instead functionally “ghrelin-deficient”, due to reduced or lack of function of said ghrelin-producing cells. In another preferred embodiment of the invention, the individual in need of treatment has an abnormally low number of ghrelin-producing cells, due e.g to damage to the epithelium of the GI tract, such as disruption of the small and/or large intestines.

In one preferred embodiment of the present invention, the individual treated is suffering from a catabolic condition. In another preferred embodiment of the present invention, the individual treated is suffering from a pathological condition associated with insulin resistance.

Another aspect of the present invention encompasses the use of a secretagogue compound for the preparation of a medicament for the prophylaxis or treatment of one or more of the following
loss of fat mass
weight loss
Loss of lean body mass
cachexia
loss of appetite
immunological dysfunction
Bone fracture (by e.g. improving the condition of the bone minerals and as supportive care)
malnutrition
disrupted sleep pattern
drowsiness
lowered intestinal absorption
intestinal motility problems
   in an individual suffering from, or at risk or suffering from, a pathological condition associated with insulin resistance. Preferably, said condition associated with insulin resistance is selected from the group consisting of: polycystitic ovary syndrome, acromegaly, primary/secondary hypogonadism, Non-AlcoholicFatty Liver Disease (NAFLD) and/or Type I Diabetes Mellitus

Another aspect of the present invention encompasses use of a secretagogue compound is used for the preparation of a medicament for the prophylaxis or treatment of one or more of the following
loss of fat mass
loss of lean body mass
weight loss
cachexia
loss of appetite
immunological dysfunction
malnutrition
disrupted sleep pattern
drowsiness
lowered intestinal absorption
intestinal motility problems
   in an individual suffering from, or at risk of suffering from, disrupted epithelium in the GI tract. Said disruption is preferably caused by chemotherapy and/or radiotherapy. Equally preferably, said disruption is caused by gastristis.

Another aspect of the present invention encompasses the use of a secretagogue compound for the preparation of a medicament for the prophylaxis or treatment of one or more of the following
loss of fat mass
Loss of lean body mass
weight loss
cachexia
loss of appetite
immunological dysfunction
malnutrition
disrupted sleep pattern
drowsiness
lowered intestinal absorption
intestinal motility problems
   in an individual suffering from, or at risk of suffering from hyperthyroidism. Preferably, said hyperthyroidism is caused by one or more of the following: Grave's disease, drugs containing a high level of iodine, thyroiditis, subacute thyroiditis, postpartum thyroiditis, loss of feedback control of thyroid hormone producing cells, toxic nodular goiter, excessive doses of thyroid hormone or thyroid medication.

“The term “malnutrition” refers to a state whereby an individual does not consume, absorb, or maintain in their body sufficient levels of one or more macro- or micro-nutrients so as to remain fit and healthy. By “immunosuppressed” is meant that the individual has a lower than average immune function. An immunosuppressed person may have, for example, a lowered white blood cell count. Causes of. are, for example, bone marrow reduction and/or reduced protein intake (one form of malnutrition): both these factors may be caused by chemotherapy.

In one aspect, the present invention is directed to the treatment of individuals being converted from a hyperthyroidic state to euthyroid state, and/or being in remission from a hyperthyroidic state, particularly those patients at risk of weight gain.

In one embodiment, the individual is suffering from, or in remission from suffering from, Grave's disease. In another embodiment, the individual is suffering from, or in remission from suffering from, thyroiditis, such as subacute thyroiditis, postpartum thyroiditis or De Quervain thyroiditis. In another embodiment, the individual is suffering from, or in remission from suffering from, solitary adenoma. In another embodiment, the individual is suffering from, or in remission from suffering from, toxic nodular goitre. In another embodiment, the individual is suffering from, or in remission from suffering from, symptoms caused by an excessive dose of thyroid hormone, for instance an individual who has taken a form of thyroid medication that contains T3.

In another embodiment, the individual is suffering from, or in remission from suffering from, symptoms caused by loss of feedback control of thyroid producing cells.

### Quality of Life

In all embodiments of the present invention, it is preferred that the treatment method and/or pharmaceutical compositions and/or compounds of the present invention are capable of affording the individual thus treated an improved quality of life (QOL), for example as is caused by improved body weight and/or nutritional status. Thus, in one aspect the invention relates to improvements of Quality of Life using a secretagogue, such as ghrelin or a ghrelin-like compound as described herein. In another embodiment, said improvement in an individual's life quality is assessed using a “Quality of life” questionnaire, as is known to one skilled in the art.

Two validated quality of life surveys preferred for use in assessing improved quality of life as caused by the administration of the compounds of the present invention are as follows:

(i) Medical Outcomes Study Short-Form Health Survey (SF-36). The SF-36 contains 36 questions that assess eight aspects of the patients' QOL; physical functioning (PF), role-physical functioning (RP), bodily pain (BP), general health (GH), vitality (VT), social functioning (SF), role emotional functioning (RE), and mental health (MH). According to the manual and interpretation guide responses to questions within scales are summed and linearly transformed to scale scores that range from 0, representing poor health status, to 100, representing optimal health status. The Swedish version has been validated and normative data have been presented for the general Swedish population (Sullivan M K J, Ware J. Hälsoenkät: svensk manual och tolkningsguide (SF-36 Health Survey. Swedish manual and interpretation guide). Göteborg: Sahigrenska University Hospital; 1994.)
(ii) EORTC QLQ-C30 (+3) questionnaire. The EORTC QLQ-C30 (version 1.0) is a 30 item core questionnaire intended for assessment of QOL among patients, the instrument is developed by the EORTC Quality of Life Study group. The first version has been validated in cancer patients and reference data from general populations have been published. The questionnaire comprises five functional scales; physical functioning (five questions), role functioning (two questions), emotional functioning (four questions), cognitive functioning (two questions) and social functioning (two questions). There are three symptom scales; fatigue (three questions), nausea and vomiting (two questions) and pain (two questions), and there are six single items on dyspnoea, insomnia, loss of appetite, constipation, diarrhea and financial difficulties. Two global questions are asking about the patient's health status and overall QOL. All scales and single-items measures range in score from 0 to 100. A high score for the functioning scales and the global health status and QOL represents a high level of functioning/health status and QOL. A high score for the symptom/item scales represents a high level of symptoms/problems. The QOL scores can be calculated according to the EORTC QLQ-C 30 scoring manual.

Preferred questionnaires for assessing a patient's improved quality of life after treatment with one or more secretagogue compounds are given in Example 8 of PCT application with publication no. WO2005014032 (Gastrotech Pharma A/S).

In preferred embodiments of the present invention, treatment of patients with the described conditions results in a significant improvement in the patients quality of life. Preferably, the treatment results in a significant increase in quality of life as measured using any method for testing the quality of life including, but not limited to, the above mentioned questionnaires, e.g. an increase in the quality of life score(s), or a composite quality of life score, as appropriate for the individual measuring tool, or a decrease in score(s) related to the symptoms and/or problems, respectively. This increase or decrease, respectively, is preferably 1% above the score obtained prior to initiation of the treatment, more preferably 2% above, even more preferred 5%, such as 10%, even more preferred 20%, 50% or 75% above the pre-treatment score. In another embodiment, the treatment results in measurable increases in quality of life score such that the score after treatment is equal to the average score found in a comparable healthy subject pool, or close to such a “normal” score, i.e. more than 50% of the score, even more preferably 60% of the score, or more preferably 75% of the score. Further, in another embodiment, the treatment results in a decrease in the score(s) related to the symptoms and/or problems of at least 1%, more preferably 3%, even more preferably 5% or more preferred 10%, 20%, 30% or 50% of the score(s) prior to initiation of treatment. These increases or reductions, respectively, may refer to one, several, or all of the aspects of the individual quality of life measuring tool, or a composite score when appropriate.

Any secretagogue, such as ghrelin or a ghrelin-like compound, may be used in the present invention. The term “secretagogue” according to the invention is used in its normal meaning, i.e. a substance capable of stimulating growth hormone release. In the present context, a secretagogue is defined by its ability of binding GHS-R 1a, and more preferably activating the receptor. The secretagogues of the present invention may be acylated or non-acylated. A preferred secretagogue for use in the present invention is a ghrelin analogue. “Ghrelin analogue” and “ghrelin-like compound” are used interchangeably herein, and are understood to refer to any peptide or non-peptide compound that essentially exerts the same biological effect as ghrelin in vivo. Exemplary non-peptide ghrelin analogues are described in EP 0 869 974 and EP 1 060 190, which illustrate a number of ghrelin analogues and which documents are incorporated herein by way of reference.

In one preferred embodiment, the ghrelin-like compound for use in the present invention includes the naturally occurring 28 aa human ghrelin, the amino acid of which is shown in SEQ ID NO: 1, as well as the naturally occurring 27 aa human ghrelin, the amino acid of which is shown in SEQ ID NO: 2.

### Ghrelin-Like Compound

Any GHS-R1A secretagogue, such as ghrelin or a ghrelin-like compound, may be used in the present invention. One preferred type of ghrelin-like compound according to the invention described herein is a compound comprising a structure defined by formula I:

Z¹-(X¹)ₘ—(X²)—(X³)ₙ-Z², wherein Formula I

Z¹ is an optionally present protecting group
each X¹ is independently selected from an amino acid, wherein said amino acid is selected from naturally occurring and synthetic amino acids,
X² is any amino acid selected from naturally occurring and synthetic occurring amino acids, said amino acid being modified with a bulky hydrophobic group, preferably an acyl group, or a fatty acid,
each X³ is independently selected from an amino acid, wherein said amino acid is selected from naturally occurring and synthetic amino acids,
wherein one or more of X¹ and X³ optionally may be modified by a bulky hydrophobic group, preferably an acyl group, or a fatty acid,
Z² is an optionally present protecting group,
m is an integer in the range of from 1-10
n is 0 or an integer in the range of from 1-35.

Accordingly, the term “secretagogue” or “growth hormone secretagogue”, or “GHS-R1a secretagogue” includes the naturally occurring 28 aa human ghrelin, the amino acid of which is shown in SEQ ID NO: 1, as well as the naturally occurring 27 aa human ghrelin, the amino acid of which is shown in SEQ ID NO: 2. Thus, the present invention relates to the use of ghrelin or a peptide homologous thereto. Ghrelin is described by Kojima in Nature (1999), vol. 402, 656-660.

The present invention includes diastereomers as well as their racemic and resolved enantiomerically pure forms. GHS-R1a secretagogues can contain D-amino acids, L-amino acids, alpha-amino acid, beta-amino acid, gamma-amino acid, natural amino acid and synthetic amino acid or the like or a combination thereof. Preferably, amino acids present in a ghrelin-like compound are the L-enantiomer.

Further suitable GHS-R1a secretagogues for use in the present invention are disclosed in PCT patent application no. PCT/DK2004/000529, Danish patent application no. PA 200401875, and PCT applications with publication numbers WO0192292 (Merck and Co. Inc), WO0134593 (Novo Nordisk AS) and WO0107475 (“Novel peptides”, Kangawa et al.); said documents all being incorporated herein by reference.

Methods for production of GHS-R1a secretagogues are well known to those skilled in the art, for example in Example 2 of PCT patent application PCT/DK2004/000519 (Gastrotech Pharma), incorporated herein by reference.

### Functionality

The GHS-R1A ligands described herein are active at the receptor for GHS as described above, i.e. the receptor GHS-R 1a. The compounds can bind to the receptor, and stimulate, partially stimulate, or inhibit receptor activity. Furthermore, the compounds may be able to modulate the activity of other GHS-R1A ligands, such as ghrelin, by for instance blocking the action of ghrelin—i.e. antagonize the effects of agonists.

Agonists of the GHS-R1A may be either full agonists, i.e. be able to fully stimulate the receptor and the signalling cascades, equal to the activities of ghrelin, or partial agonists, i.e. ligands that are only able to partially stimulate the receptor and the signalling cascade, measured as described below. Such partial agonists may also be able to fully or partially antagonize the actions of full agonists such as ghrelin.

The receptor activity can be measured using different techniques such as detecting a change in the intracellular conformation of the receptor, in the activity of the G-protein coupled to the receptor, and/or in alteration of the level of intracellular messengers.

One simple measure of the ability of a ligand to activate the ghrelin receptor is to measure its EC50, i.e. the dose at which the compound activates the receptor to half of the maximal obtainable effect using same compound. The receptor can either be expressed endogenously on primary cells cultures, for example pituitary cells, or heterologously expressed on cells transfected with a cDNA encoding the ghrelin receptor. Whole cell assays or assays using membranes prepared form either of these cell types can be used depending on the type of assay.

As the receptor is generally believed to be primarily coupled to the Gq signalling pathway, any suitable assay which monitors activity in the Gq/G11 signalling pathway can be used, for example:
1) an assay measuring the activation of Gq/G11 performed for example by measurement of GTPgS binding combined with, e.g., anti-G-alpha-q or -11 antibody precipitation in order to increase the signal to noise ratio. This assay may also detect coupling to other G-proteins than Gq/11.
2) An assay which measure the activity of phopholipase C (PLC) one of the first down-stream effector molecules in the pathway, for example by measuring the accumulation of inositol phosphate which is one of the products of PLC.
3) More down stream in the signalling cascade is the mobilization of calcium from the intracellular stores
4) Further more down stream signalling molecules such as the activity of different kinds of MAP kinases (ERK ½, p38, junK, etc.). NF-κ-B translocation and CRE driven gene transcription may also be measured.
5) Binding of fluorescently tagged arrestin to the activated ghrelin receptor

Examples of suitable protocols for use in determining GHS-R1A ligand functionality are given in Example 5 of PCT application publication no. WO2005014032 (Gastrotech Pharma A/S).

In one embodiment the binding of a compound to the receptor GHS-R 1A is measured by the use of any of the assays described herein above.

A GHS-R1A ligand according to the invention preferably has at least about 50%, at least about 60%, at least about 70%, at least about 80%, or at least about 90%, functional activity relative to 28 aa acylated human ghrelin as determined using the assay described herein above. Greater refers to potency and thus indicates a lesser amount is needed to achieve binding inhibition.

In one embodiment of the invention, the GHS-R1A ligand has a potency (EC50) on the GHS-R 1A of less than 500 nM. In another embodiment the compound has a potency (EC50) on the GHS-R 1A of less than 100 nM, such as less than 80 nM, for example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

In a further embodiment the dissociation constant (Kd) of the GHS-R1A ligand is less than 500 nM. In a still further embodiment the dissociation constant (Kd) of the ligand is less than 100 nM, such as less than 80 nM, for example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

Binding assays can be performed using recombinantly-produced receptor polypeptides present in different environments. Such environments include, for example, cell extracts and purified cell extracts containing the receptor polypeptide expressed from recombinant nucleic acid or naturally occurring nucleic acid; and also include, for example, the use of a purified GHS receptor polypeptide produced by recombinant means or from naturally occurring nucleic acid which is introduced into a different environment.

Using a recombinantly expressed GHS receptor offers several advantages such as the ability to express the receptor in a defined cell system, so that a response to a compound at the receptor can more readily be differentiated from responses at other receptors. For example, the receptor can be expressed in a cell line such as HEK 293, COS 7, and CHO not normally expressing the receptor by an expression vector, wherein the same cell line without the expression vector can act as a control.

### Identity and Homology

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

A homologue of one or more of the sequences specified herein may vary in one or more amino acids as compared to the sequences defined, but is capable of performing the same function, i.e. a homologue may be envisaged as a functional equivalent of a predetermined sequence. A ghrelin homologue is preferably a ghrelin-like compound as defined above.

As described above a homologue of any of the predetermined sequences herein may be defined as:
i) homologues comprising an amino acid sequence capable of being recognised by an antibody, said antibody also recognising the 28 aa human ghrelin, preferably the acylated 28 aa human ghrelin, and/or
ii) homologues comprising an amino acid sequence capable of binding selectively to GHS-R 1a, and/or
iii) homologues having a substantially similar or higher binding affinity to GHS-R 1a than the 28 aa human ghrelin, preferably the acylated 28 aa human ghrelin.

In the above examples, the 28 aa human ghrelin has the sequence shown in SEQ ID NO:1, and when acylated is acylated in position 3.

The antibodies used herein may be antibodies binding the N-terminal part of ghrelin or the C-terminal part of ghrelin, preferably the N-terminal part of ghrelin. The antibodies may be antibodies as described in Ariyasu et al. “Delayed short-term secretory regulation of ghrelin in obese animals: Evidensed by a specific RIA for the active form of ghrelin, Endocrinology 143(9):3341-3350, 2002.

Examples of homologues comprises one or more conservative amino acid substitutions including one or more conservative amino acid substitutions within the same group of predetermined amino acids, or a plurality of conservative amino acid substitutions, wherein each conservative substitution is generated by substitution within a different group of predetermined amino acids.

Homologues may thus comprise conservative substitutions independently of one another, wherein at least one glycine (Gly) of said homologue is substituted with an amino acid selected from the group of amino acids consisting of Ala, Val, Leu, and Ile, and independently thereof, homologues, wherein at least one of said alanines (Ala) of said homologue thereof is substituted with an amino acid selected from the group of amino acids consisting of Gly, Val, Leu, and Ile, and independently thereof, homologues, wherein at least one valine (Val) of said homologue thereof is substituted with an amino acid selected from the group of amino acids consisting of Gly, Ala, Leu, and Ile, and independently thereof, homologues thereof, wherein at least one of said leucines (Leu) of said homologue thereof is substituted with an amino acid selected from the group of amino acids consisting of Gly, Ala, Val, and Ile, and independently thereof, homologues thereof, wherein at least one isoleucine (Ile) of said homologues thereof is substituted with an amino acid selected from the group of amino acids consisting of Gly, Ala, Val and Leu, and independently thereof, homologues thereof wherein at least one of said aspartic acids (Asp) of said homologue thereof is substituted with an amino acid selected from the group of amino acids consisting of Glu, Asn, and Gln, and independently thereof, homo logues thereof, wherein at least one of said phenylalanines (Phe) of said homo logues thereof is substituted with an amino acid selected from the group of amino acids consisting of Tyr, Trp, His, Pro, and preferably selected from the group of amino acids consisting of Tyr and Trp, and independently thereof, homologues thereof, wherein at least one of said tyrosines (Tyr) of said homologues thereof is substituted with an amino acid selected from the group of amino acids consisting of Phe, Trp, His, Pro, preferably an amino acid selected from the group of amino acids consisting of Phe and Trp, and independently thereof, homologues thereof, wherein at least one of said arginines (Arg) of said fragment is substituted with an amino acid selected from the group of amino acids consisting of Lys and His, and independently thereof, homologues thereof, wherein at least one lysine (Lys) of said homologues thereof is substituted with an amino acid selected from the group of amino acids consisting of Arg and His, and independently thereof, homologues thereof, wherein at least one of said aspargines (Asn) of said homologues thereof is substituted with an amino acid selected from the group of amino acids consisting of Asp, Glu, and Gln, and independently thereof, homologues thereof, wherein at least one glutamine (Gln) of said homologues thereof is substituted with an amino acid selected from the group of amino acids consisting of Asp, Glu, and Asn, and independently thereof, homologues thereof, wherein at least one proline (Pro) of said homologues thereof is substituted with an amino acid selected from the group of amino acids consisting of Phe, Tyr, Trp, and His, and independently thereof, homologues thereof, wherein at least one of said cysteines (Cys) of said homologues thereof is substituted with an amino acid selected from the group of amino acids consisting of Asp, Glu, Lys, Arg, His, Asn, Gln, Ser, Thr, and Tyr.

Conservative substitutions may be introduced in any position of a preferred predetermined sequence. It may however also be desirable to introduce non-conservative substitutions, particularly, but not limited to, a non-conservative substitution in any one or more positions.

The following table lists preferred, but non-limiting, conservative amino acid substitutions.

| ORIGINAL RESIDUE | EXEMPLARY SUBSTITUTIONS |
|---|---|
| ALA | SER, THR, VAL, GLY |
| ARG | LYS |
| ASN | HIS, SER |
| ASP | GLU, ASN |
| CYS | SER |
| GLN | ASN, HIS |
| GLU | ASP, GLU |
| GLY | ALA, SER |
| HIS | ASN, GLN |
| ILE | LEU, VAL, THR |
| LEU | ILE, VAL |
| LYS | ARG, GLN, GLU, THR |
| MET | LEU, ILE, VAL |
| PHE | LEU, TYR |
| SER | THR, ALA, ASN |
| THR | SER, ALA |
| TRP | ARG, SER |
| TYR | PHE |
| VAL | ILE, LEU, ALA |
| PRO | ALA |

A non-conservative substitution leading to the formation of a functionally equivalent homologue of the sequences herein would for example i) differ substantially in polarity, for example a residue with a non-polar side chain (Ala, Leu, Pro, Trp, Val, lie, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In a preferred embodiment the binding domain comprises a homologue having an amino acid sequence at least 60% homologous to SEQ ID NO 1.

More preferably the homology is at least 70%, such as at least 75% homologous, such as at least 80% homologous, such as at least 85% homologous, such as at least 90% homologous, such as at least 95% homologous, such as at least 97% homologous, such as at least 98%, for example at least 99% homologous to SEQ ID NO 1.

In a more preferred embodiment the percentages mentioned above relates to the identity of the sequence of a homologue as compared to SEQ ID NO 1.

Homologues to SEQ ID NO: 1 may be 27 aa human ghrelin SEQ ID NO: 2, rat ghrelin SEQ ID NO: 3. Other homologues are the variants described in EP 1197496 (Kangawa) incorporated herein by reference.

### Bulky Hydrophobic Group

The bulky hydrophobic group of the secretagogue according to the invention is any bulky hydrophobic group capable of providing the des-acylated 28 aa human ghrelin with binding affinity to GHS-R 1a when the Ser residue in position 3 is modified with the bulky hydrophobic group.

When the amino acid being modified contains e.g. —OH, —SH, —NH or —NH₂ as a substituent group in a side chain thereof, a group formed by acylating such a substituent group is preferred. The mode of linkage may thus be selected from the group consisting of ester, ether, thioester, thioether, amide and carbamide.

For example, if the modified amino acid is serine, threonine, tyrosine or oxyproline, the amino acid has a hydroxyl group in the side chain. If the modified amino acid is cysteine, the amino acid has a mercapto group in the side chain. If the modified amino acid is lysine, arginine, histidine, tryptophan, proline or oxyproline, it has an amino group or imino group in the side chain.

The hydroxyl group, mercapto group, amino group and imino group described above may thus have been chemically modified. That is, the hydroxyl group or mercapto group may be etherized, esterified, thioetherified or thioesterified. The imino group may have been iminoetherified, iminothioetherified or alkylated. The amino group may have been amidated, thioamidated or carbamidated.

Further, the mercapto group may have been disulfidated, the imino group may have been amidated or thioamidated, and the amino group may have been alkylated or thiocarbamidated.

In a preferred embodiment the modified amino acid is Ser coupled through an ester linkage to the hydrophobic group.

The hydrophobic group may be any group with a saturated or unsaturated alkyl or acyl group containing one or more carbon atoms. In one embodiment the bulky hydrophobic group is an acyl group, including groups formed by removing a hydroxyl group from an organic carboxylic acid, organic sulfonic acid or organic phosphoric acid. The organic carboxylic acid includes e.g. fatty acids, and the number of carbon atoms thereof is preferably 1 to 35. In the organic sulfonic acid or organic phosphoric acid, the number of carbon atoms thereof is preferably 1 to 35.

Accordingly, the acyl group is preferably selected from a C1-C35 acyl group, such as a C1-C20 acyl group, such as a C1-C15 acyl group, such as a C6-C15 acyl group, such as a C6-C12 acyl group, such as a C8-C12 acyl group.

More preferably the acyl group is selected from the group of C7 acyl group, C8 acyl group, C9 acyl group, C10 acyl group, C11 acyl group, and C12 acyl group. Such acyl group may be formed from octanoic acid (preferably caprylic acid), decanoic acid (preferably capric acid), or dodecanoic acid (preferably lauric acid), as well as monoene or polyene fatty acids thereof.

In one embodiment the acyl group is selected from the group of C8 acyl group, and C10 acyl group. Such acyl groups may be formed from octanoic acid (preferably caprylic acid), or decanoic acid (preferably capric acid).

In another embodiment the acyl group is selected from the group of C7 acyl group, C9 acyl group, and C11 acyl group, such as from the group of C9 acyl group and C11 acyl group.

Furthermore, the modified amino acid may be any amino acid wherein a group is modified as described in EP 1 197 496 (Kangawa), which is hereby incorporated by reference.

### Protecting Group

The secretagogue according to the invention may comprise a protecting group at the N-terminus or the C-terminus or at both.

A protecting group covalently joined to the N-terminal amino group reduces the reactivity of the amino terminus under in vivo conditions. Amino protecting groups include —C1-10 alkyl, —C1-10 substituted alkyl, —C2-10 alkenyl, —C2-10 substituted alkenyl, aryl, —C1-6 alkyl aryl, —C(O)— (CH2) 1-6—COOH, —C(O)—C1-6 alkyl, —C(O)-aryl, —C(O)—O—C1-6 alkyl, or —C(O)—O-aryl. Preferably, the amino terminus protecting group is acetyl, propyl, succinyl, benzyl, benzyloxycarbonyl or tbutyloxycarbonyl.

A protecting group covalently joined to the C-terminal carboxy group reduces the reactivity of the carboxy terminus under in vivo conditions. The carboxy terminus protecting group is preferably attached to the a-carbonyl group of the last amino acid. Carboxy terminus protecting groups include amide, methylamide, and ethylamide.

### Conjugates

The secretagogue, such as a ghrelin-like compound, to be used in the present invention may be provided in the form of a secretagogue conjugate, i.e. a molecule comprising the secretagogue conjugated to another entity, for example in order to prolong its half-life. The other entity may be any substance that is capable of conferring improved properties to the secretagogue, e.g. in terms of improved stability, half-life, etc.

In one embodiment the conjugate is a conjugate of ghrelin or a derivative or homologue thereof and Ac-RYY(RK)(WI)RK)—NH₂, where the brackets show allowable variation of amino acid residues. Examples of peptides in the conjugate may also be found in US patent application 2003040472.

### Pharmaceutical Compositions

Whilst it is possible for the compounds or salts of the present invention to be administered as the raw chemical, it is preferred to present them in the form of a pharmaceutical composition. Accordingly, the present invention provides pharmaceutical compositions useful for practising the therapeutic methods described herein. Said pharmaceutical compositions preferably contain a physiologically tolerable carrier together with at least one species of a secretagogue, such as ghrelin or a ghrelin-like compound as described herein (such as a compound as defined above in formula I), or salt thereof, dissolved or dispersed therein as an active ingredient. Said compositions of the present invention may preferably be delivered to an individual in any way so as to achieve a beneficial effect, preferably by stimulating appetite and/or preventing malnutrition, and/or improving the individual's sense of well-being or quality of life. In one preferred embodiment, a composition according to the present invention is administered via an oral, nasal, pulmonary, transdermal or parenteral route. More preferably, the composition is administered via the oral or pulmonary route. In another preferred embodiment, said administration is subcutaneous. Other drug-administration methods, which are effective to deliver the drug to a target site or to introduce the drug into the bloodstream, are also contemplated.

The compounds according to the invention may be administered with at least one other compound. The compounds may be administered simultaneously, either as separate compositions or combined in a unit dosage form, or administered sequentially. In one particular embodiment the invention relates to the use of a pharmaceutical composition comprising a mixture of at least two different ghrelin-like compounds, such as a mixture of a ghrelin-like compound being acylated with a C8 acyl and a ghrelin-like compound being acylated with a C10 acyl. Without being bound by theory it is believed that such a mixture will have a longer half-life in plasma. Thus, in a preferred embodiment the pharmaceutical composition comprises at least two different ghrelin-like compounds as defined above in formula I in order to increase the effect of the treatment. The difference may for example be compounds having different acylations as discussed above.

In yet another embodiment, the pharmaceutical composition used comprises acylated ghrelin-like compounds, optionally compounds having different acyl chain lengths preferably selected from the group of C7 acyl group, C9 acyl group, and C11 acyl group, such as from the group of C9 acyl group and C11 acyl group, further optionally in combination with a desacylated Ghrelin-like compound.

In a preferred embodiment, the pharmaceutical composition is not immunogenic when administered to a individual for therapeutic purposes, unless that purpose is to induce an immune response.

Preferably, the composition comprises ghrelin or an analogue or pharmaceutically acceptable salt thereof and pharmaceutically acceptable carriers, vehicles and/or excipients and/or transport molecules, such as for the treatment of loss of body weight and body fat in an individual subjected to chemotherapeutic treatment.

The transport molecules are primarily added in order to increase the half-life of the acylated compound, preventing premature des-acylation, since the des-acylated ghrelin is not active at the GHS-R1a. Transport molecules act by having incorporated into or anchored to it the compound according to the invention. Any suitable transport molecules known to the skilled person may be used. Examples of transport molecules are those described in the conjugate section. Other preferred examples are liposomes, micelles, and/or microspheres.

Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipid or modifiers of liposomes are preferred e.g. in combination with cholesterol and/or phosphatidylcholine.

Long-circulating liposomes are characterized by their ability to extravasate at body sites where the permeability of the vascular wall is increased. The most popular way to produce long circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N—[Methoxy(Polyethylene glycol)-5000] (Ammonium Salt), 1,2-Dioleoyl-3-Trimethylammonium-Propane (Chloride Salt) (DOTAP).

Possible lipid applicable for liposomes are supplied by Avanti, Polar lipids, Inc, Alabaster, Ala. Additionally, the liposome suspension may include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference.

One suitable method for preparing liposomes is prepared in Example 9 of PCT application with publication no. WO2005014032 (Gastrotech Pharma A/S), which is validated in example 10 of the same application as being capable of increasing plasma levels of ghrelin. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder like form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Micelles are formed by surfactants (molecules that contain a hydrophobic portion and one or more ionic or otherwise strongly hydrophilic groups) in aqueous solution. As the concentration of a solid surfactant increases, its monolayers adsorbed at the air/water or glass/water interfaces become so tightly packed that further occupancy requires excessive compression of the surfactant molecules already in the two monolayers. Further increments in the amount of dissolved surfactant beyond that concentration cause amounts equivalent to the new molecules to aggregate into micelles. This process begins at a characteristic concentration called “critical micelle concentration”.

The shape of micelles formed in dilute surfactant solutions is approximately spherical. The polar head groups of the surfactant molecules are arranged in an outer spherical shell whereas their hydrocarbon chains are oriented toward the center, forming a spherical core for the micelle. The hydrocarbon chains are randomly coiled and entangled and the micellar interior has a nonpolar, liquid-like character. In the micelles of polyoxyethylated nonionic detergents, the polyoxyethlene moieties are oriented outward and permeated by water. This arrangement is energetically favorable since the hydrophilic head groups are in contact with water and the hydrocarbon moieties are removed from the aqueous medium and partly shielded from contact with water by the polar head groups. The hydrocarbon tails of the surfactant molecules, located in the interior of the micelle, interact with one another by weak van der Waals forces.

The size of a micelle or its aggregation number is governed largely by geometric factors. The radius of the hydrocarbon core cannot exceed the length of the extended hydrocarbon chain of the surfactant molecule. Therefore, increasing the chain length or ascending homologous series increases the aggregation number of spherical micelles. If the surfactant concentration is increased beyond a few percent and if electrolytes are added (in the case of ionic surfactants) or the temperature is raised (in the case of nonionic surfactants), the micelles increase in size. Under these conditions, the micelles are too large to remain spherical and become ellipsoidal, cylindrical or finally lamellar in shape.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-.beta.-D-glucopyranoside, and the like. In addition, phospholipids, such as those described for use in the production of liposomes, may also be used for micelle formation.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon an individual without the production of undesirable physiological effects such as nausea, dizziness, gastric upset and the like.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. It is preferred that the formulation has a pH within the range of 3.5-8, such as in the range 4.5-7.5, such as in the range 5.5-7, such as in the range 6-7.5, most preferably around 7.3. However, as is understood by one skilled in the art, the pH range may be adjusted according to the individual treated and the administration procedure. For example, certain secretagogues, such as ghrelin and ghrelin homologs, may be easily stabilised at a lower pH, so in another preferred embodiment of the invention the formulation has a pH within the range 3.5-7, such as 4-6, such as 5-6, such as 5.3-5.7, such as 5.5.

The pharmaceutical composition of the present invention can include pharmaceutically acceptable salts of the compounds therein. Pharmaceutically acceptable salts include the acid addition salts (formed with the free amino groups of the polypeptide).

Such salts include pharmaceutically acceptable acid addition salts, pharmaceutically acceptable metal salts, ammonium salts and alkylated ammonium salts. Acid addition salts include salts of inorganic acids as well as organic acids. Representative examples of suitable inorganic acids include hydrochloric, hydrobromic, hydriodic, phosphoric, sulphuric and nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, ethylenediaminetetraacetic (EDTA), p-aminobenzoic, glutamic, benzenesulfonic and ptoluenesulfonic acids and the like. Further examples of pharmaceutically acceptable inorganic or organic acid addition salts include the pharmaceutical acceptable salts listed in J. Pharm. Sci. 1977,66,2, which is incorporated herein by reference. Examples of metal salts include lithium, sodium, potassium and magnesium salts and the like.

Examples of ammonium and alkylated ammonium salts include ammonium, methylammonium, dimethylammonium, trimethylammonium, ethylammonium, hydroxyethylammonium, diethylammonium, butylammonium and tetramethylammonium salts and the like.

Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Also included within the scope of compounds or pharmaceutical acceptable acid addition salts thereof in the context of the present invention are any hydrates (hydrated forms) thereof.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. For e.g. parenteral administration, solutions of the present compounds in sterile aqueous solution, aqueous propylene glycol or sesame or peanut oil may be employed. Such aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art. Formulations suitable for administration by e.g. nasal aerosols or inhalation, formulations may be prepared, for example, as solutions in saline, employing benzyl alcohol or other suitable preservatives, absorption promoters to enhance bioavailability, employing fluorocarbons, and/or employing other solubilizing or dispersing agents.

The pharmaceutical compositions formed by combining the compounds of the invention and the pharmaceutical acceptable carriers are then readily administered in a variety of dosage forms suitable for the disclosed routes of administration. The compositions may conveniently be presented in unit dosage form by methods known in the art of pharmacy.

In a preferred embodiment of the invention the composition comprises the GH secretagogue or a salt thereof as a lyophilisate and the composition further comprises a solvent. In another embodiment the composition is a solution of the secretagogue or a salt thereof. Preferably, the solvent may be any suitable solvents, such as described herein, and preferably the solvent is saline or a physiological buffer like phosphate buffer.

The invention also relates to a method for preparing a medicament or pharmaceutical composition comprising an compound of the invention, comprising admixing at least one GH secretagogue as defined above with a physiologically acceptable carrier.

In a still further aspect, the invention relates to a pharmaceutical composition comprising, as an active ingredient, a compound as defined above or a pharmaceutical acceptable salt thereof together with a pharmaceutical acceptable carrier.

Accordingly, the composition may further include the transport molecules as described above.

### Administration

Preferred compositions useful in the present invention contain the active ingredient together with a pharmaceutically acceptable carrier or diluent, which can be selected by the skilled artisan according to the route of administration. The pharmaceutical carrier or diluent employed may be a conventional solid or liquid carrier, e.g. lactose, cyclodextrin, talc, gelatin, agar, pectin, magnesium stearate, cellulose-derivatives, or syrup, olive oil, phospholipids, polyoxyethylene or simply water. Similarly, the carrier or diluent may include any sustained release material known in the art, such as glyceryl monostearate or glyceryl distearate, alone or admixed with one or more waxes. The compositions may appear in conventional forms, such as capsules, tablets, aerosols, solutions, suspensions or topical applications. As described herein, the formulation may also comprise liposomes and/or micelles.

For the present indication the dosage will vary depending on the compound employed and the mode of administration. Dosage levels will vary between about 0.01 μg/kg body weight to 10 g/kg body weight daily, preferably between about 0.01 μg/kg body weight to 1 mg/kg body weight, more preferably between 0.01 to 10 μg/kg body weight, most preferably about 0.01 μg/kg body weight The route of administration may be any route which effectively transports the active compound to the appropriate or desired site of action, such as oral, nasal, pulmonary, transdermal or parenteral, the oral or pulmonar route being preferred.

The objective compounds may be administered as a pharmaceutically acceptable acid addition salt or, where appropriate, as a alkali metal or alkaline earth metal or lower alkylammonium salt. Such salt forms are believed to exhibit approximately the same order of activity as the free base forms. Suitable dosages may for example range from about 50 mg to about 200 mg, preferably from about 20 mg to about 100 mg of the compounds of formula I admixed with a pharmaceutically acceptable carrier or diluent. In another preferred embodiment, a suitable dosage is 10 μg/kg, preferably administered once daily.

Any administration form that will ensure that the ghrelin receptors which normally are the target for peripherally produced ghrelin will be exposed to sufficient levels of the bioactive form of ghrelin (or another secretagogue) may be part of the present invention. However, taken into consideration that the individuals to be treated possibly will have to receive treatment for a longer period, such as weeks or months, it is preferred that the administration form is well suited therefore.

In one embodiment, it is preferred that the secretagogue, such as a ghrelin-like compound, is to be administered to an individual in need thereof in an amount so as to generate a concentration of secretagogue that is at least functionally equivalent to a non-ghrelin deficient individual's ghrelin levels, such as to generate a concentration of secretagogue that is functionally equivalent to a non-ghrelin deficient individual's ghrelin levels. The functionality of the various secretagogue compounds described herein may be assayed using any of the methods described herein.

Accordingly, it is preferred that the secretagogue, such as a ghrelin-like compound, according to the invention is administered subcutaneously in an amount sufficient to allow sufficient levels of the bioactive form of ghrelin, i.e. the acylated form, to reach the receptors. An example showing the efficacy of subcutaneous administration of ghrelin is given in Example 6 of PCT application with publication no. WO2005014032 (Gastrotech Pharma A/S).

One embodiment of the present invention preferably deals with methods for administering a secretagogue, such as ghrelin, in a way which mimics the physiologically pre-meal situation as closely as possible.

As described above, in one aspect of the invention, the secretagogue, such as ghrelin or a ghrelin-like compound, is administered subcutaneously.

In another aspect the secretagogue, such as ghrelin or a ghrelin-like compound, is administered as a bolus, wherein the administration form may be any suitable parenteral form.

In a preferred embodiment the secretagogue, such as ghrelin or a ghrelin-like compound, is administered subcutaneously in a bolus.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use.

Other suitable administration forms include suppositories, sprays, ointments, cremes, gels, inhalants, dermal patches, implants, pills, tablets, lozenges and capsules.

A typical dosage of a compound employed according to the invention is in a concentration equivalent to from 10 ng to 10 mg ghrelin per kg bodyweight. The concentrations and amounts herein are given in equivalents of amount ghrelin, wherein the ghrelin is the 28 aa human ghrelin. Equivalents may be tested as described in the section entitled “Functionality”, above.

In a preferred embodiment the medicament is administered in a concentration equivalent to from 0.1 μg to 1 mg ghrelin per kg bodyweight, such as from 0.5 μg to 0.5 mg ghrelin per kg bodyweight, such as from 1.0 μg to 0.1 mg ghrelin per kg bodyweight, such as from 1.0 μg to 50 μg ghrelin per kg bodyweight, such as from 1.0 μg to 10 μg ghrelin per kg bodyweight.

As described above, the secretagogue, such as ghrelin or a ghrelin-like compound, is preferably administered as a bolus, such as a bolus comprising an amount of the secretagogue or a salt thereof equivalent to from 0.3 μg to 600 mg ghrelin; more preferably, said bolus comprises an amount of the secretagogue or a salt thereof equivalent to from 2.0 μg to 200 mg ghrelin, such as from 5.0 μg to 100 mg ghrelin, such as from 10 μg to 50 mg ghrelin, such as from 10 μg to 5 mg ghrelin, such as from 10 μg to 1.0 mg ghrelin.

### Compositions for Oral Administration

Those secretagogue types capable of remaining biologically active in an individual after oral administration (such as e.g. small molecules and short peptides) can be formulated in a wide range of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably containing from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the composition of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

Other forms suitable for (e.g. oral) administration include liquid form preparations including emulsions, syrups, elixirs, aqueous solutions, aqueous suspensions, toothpaste, gel dentifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilizers, buffers, artificial and natural sweeteners, dispersants, thickeners, solubilizing agents, and the like.

### Compositions for Parenteral Administration

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water. Aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Solutions of ghrelin or a ghrelin-like compound or pharmaceutically acceptable salt thereof, (and for example antigenic epitopes and protease inhibitors) can be prepared in water or saline, and optionally mixed with a nontoxic surfactant. Compositions for intravenous or intra-arterial administration may include sterile aqueous solutions that may also contain buffers, liposomes, diluents and other suitable additives.

Oils useful in parenteral compositions include petroleum, animal, vegetable, or synthetic oils. Specific examples of oils useful in such compositions include peanut, soybean, sesame, cottonseed, corn, olive, petrolatum, and mineral. Suitable fatty acids for use in parenteral compositions include oleic acid, stearic acid, and isostearic acid. Ethyl oleate and isopropyl myristate are examples of suitable fatty acid esters.

Suitable soaps for use in parenteral compositions include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral compositions typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such compositions will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral compositions can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The pharmaceutical dosage forms suitable for injection or infusion can include sterile aqueous solutions or dispersions comprising the active ingredient that are adapted for administration by encapsulation in liposomes. In all cases, the ultimate dosage form must be sterile, fluid and stable under the conditions of manufacture and storage.

Sterile injectable solutions are prepared by incorporating ghrelin or a ghrelin-like compound or pharmaceutically acceptable salt thereof in the required amount in the appropriate solvent with various of the other ingredients enumerated above, as required, followed by filter sterilization.

An example of a randomized, single centre, four-period cross-over trial to investigate the absolute bioavailability of iv administered Ghrelin and sc administered Ghrelin at three different single doses in healthy subjects is given in Example 3 of PCT application with publication no. WO2005014032 (Gastrotech Pharma A/S).

### Compositions for Topical Administration

The compounds of the invention can also be delivered topically. Regions for topical administration include the skin surface and also mucous membrane tissues of the rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, cream, foam, aerosol, spray, suppository, implant, inhalant, tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Compositions suitable for topical administration in the mouth include lozenges comprising active agents in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Creams, ointments or pastes according to the present invention are semi-solid compositions of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The composition may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin. Lotions or liniments for application to the skin may also include an agent to hasten drying and to cool the skin, such as an alcohol or acetone, and/or a moisturizer such as glycerol or an oil such as castor oil or arachis oil.

The pharmaceutical agent-chemical modifier complexes described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

Transdermal delivery is accomplished by exposing a source of the complex to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

A variety of types of transdermal patches will find use in the methods described herein. For example, a simple adhesive patch can be prepared from a backing material and an acrylate adhesive. The pharmaceutical agent-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Alternatively, a polyurethane matrix patch can be employed to deliver the pharmaceutical agent-chemical modifier complex. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and complex to the prepolymer results in the formation of a tacky firm elastomer that can be directly cast only the backing material.

A further embodiment of this invention will utilize a hydrogel matrix patch. Typically, the hydrogel matrix will comprise alcohol, water, drug, and several hydrophilic polymers. This hydrogel matrix can be incorporated into a transdermal patch between the backing and the adhesive layer.

The liquid reservoir patch will also find use in the methods described herein. This patch comprises an impermeable or semipermeable, heat sealable backing material, a heat sealable membrane, an acrylate based pressure sensitive skin adhesive, and a siliconized release liner. The backing is heat sealed to the membrane to form a reservoir which can then be filled with a solution of the complex, enhancers, gelling agent, and other excipients.

Foam matrix patches are similar in design and components to the liquid reservoir system, except that the gelled pharmaceutical agent-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like. The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin. Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

Other items may be contained in the device, such as other conventional components of therapeutic products, depending upon the desired device characteristics. For example, the compositions according to this invention may also include one or more preservatives or bacteriostatic agents, e.g., methyl hydroxybenzoate, propyl hydroxybenzoate, chlorocresol, benzalkonium chlorides, and the like. These pharmaceutical compositions also can contain other active ingredients such as antimicrobial agents, particularly antibiotics, anesthetics, analgesics, and antipruritic agents.

### Compositions for Administration as Suppositories

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

### Combinations

In a further aspect of the invention the present compounds may be administered in combination with further pharmacologically active substances or therapeutic method or other pharmacologically active material. By the phrase “in combination” with another substance(s) and/or therapeutic method(s) is meant herein that said another substance(s) and/or therapeutic method(s) is administered to the individual thus treated before, during (including concurrently with—preferably co-formulated with) and/or after treatment of an individual with a secretagogue. In all cases of combination treatment described herein, the combination may be in the form of kit-in-part systems, wherein the combined active substances may be used for simultaneous, sequential or separate administration. In all cases, it is preferred that any of the herein-mentioned medicaments are administered in pharmaceutically effective amounts, i.e. an administration involving a total amount of each active component of the medicament or pharmaceutical composition or method that is sufficient to show a meaningful patient benefit.

In a preferred embodiment, the secretagogue is administered to the individual with one or more medicament(s) for treatment of hyperthyroidism. Preferably, the medicament for treatment of hyperthyroidism is one or more of:
A) antithyroid drugs and/or
B) surgery and/or
C) radioiodine;
   optionally in combination with drugs targeting the cardiovascular system.

Thus, it is envisaged that the compounds of the present invention may be administered to an individual in combination with one or more other medical treatment. Said other medical treatment may comprise administering another compound or may comprise a method such as chemotherapy and/or radiotherapy.

Again, by “in combination” is mean that said other medical treatment may be carried out on said patient before, concurrently with or after administration of the compounds of the present invention. A combination may be, for example, in the form of a kit-in-part system, wherein the combined active substances may be used for simultaneous, sequential or separate administration.

In one preferred embodiment, in any of the treatments described herein, ghrelin or an analogue thereof may be used in combination with one or more other stomach-derived factor. This other stomach-derived factor may include any hormone, acylated or nonacylated peptide, amino acid derivative, nucleotide, fatty acid derivative, carbohydrate or other substance derived or secreted from the stomach, and may preferably (but not exclusively) be selected from the following list: pacreastatin, gastrin, resistine, prostaglandins such as prostaglandin E2 and intrinsic factor.

In addition to “stomach derived factors”, ghrelin can also be used in combination with any synthetic low or high molecular weight agonist acting on the same receptor as a “stomach derived factor”, such as another secretagogue.

In addition, ghrelin and/or its analogues may be used in combination with another body weight and/or body fat inducing factor. Exemplarily mentioned factors are melanin-concentrating hormone (MCH), MCH receptors agonists, especially MCH receptor 1 agonists, neuropeptide Y (NPY), NPY receptor 1 agonists, NPY receptor 5 agonists, and NPY receptor 2 antagonists including peptide YY (PYY) and PYY (3-36), alpha-melanocyte stimulating hormone (alpha-MSH, alpha-melanocortin), melanocortin-3 receptor (MC3R) antagonists, melanocortin-4 receptor (MC4R) antagonists, agouti-related peptide (Agrp), Agrp- agonists, cocaine- and amphetamine-regulated transcript (CART) antagonists, orexin receptor 1 and receptor 2 agonists, growth hormone (GH), GH receptor agonists, insulin-like growth factor-1 (IGF-1), and IGF-1 receptor 1 agonists, hypercaloric feeding, glucocorticoids, progestational drugs, cyproheptadine and other antiserotonergic drugs, branched-chain amino acids, prokinetic Agents (Motilin, metoclopramide, 10 mg), eicosapentanoic acid, cannabinoids, 5′-Deoxy-5-Fluorouridine, melatonin, thalidomide, ACE inhibitors and/or beta-receptor antagonists. Further ghrelin may be combined with agents used in the treatment of an underlying disease including antithyroid agents such as iodine, ¹³¹Iodine, propylthiouracil, thiamazole, carbimazole, methimazole, antidiabetic agents such as insulin, sulfonylureas, metformin, acarbose, thiazolidinediones, meglitinides, antacids, H2-blockers or proton pump inhibitors.

In another embodiment the GH secretagogue is administered in combination with a NSAID, such as indomethacin, and COX1 inhibitors or COX2 inhibitors. Another combination may be with erythropoietin/EPO. Another combination may be with one or more of leptin, agonists of the renin-angiotensin system, opioid receptor agonists or peroxisome proliferator-activated receptor gamma agonists. In another preferred embodiment, the secretagogue may be administered in combination with a growth hormone, preferably hGH.

Other preferred compounds or treatments for use in combination with the compounds of the present invention include one or more of: hypercaloric feeding, glucocorticoids, progestational drugs, Cyproheptadine and/or other antiserotonergic drugs, branched-chain amino acids, prokinetic agents (such as Motilin, metoclopramide, 10 mg), eicosapentanoic acid, cannabinoids, 5′-Deoxy-5-Fluorouridine, melatonin, Thalidomide and/or beta-2-agonists.

In another preferred embodiment, the GH secretagogue is administered in combination with one or more of the following: propylthiouracil, and/or methimazole and/or carbimazole.

### Medical Packaging

The compounds used in the invention may be administered alone or in combination with pharmaceutically acceptable carriers or excipients, in either single or multiple doses. The formulations may conveniently be presented in unit dosage form by methods known to those skilled in the art.

It is preferred that the compounds according to the invention are provided in a kit. Such a kit typically contains an active compound in dosage forms for administration. A dosage form contains a sufficient amount of active compound such that a desirable effect can be obtained when administered to a subject.

Thus, it is preferred that the medical packaging comprises an amount of dosage units corresponding to the relevant dosage regimen. Accordingly, in one embodiment, the medical packaging comprises a pharmaceutical composition comprising a compound as defined above or a pharmaceutically acceptable salt thereof and pharmaceutically acceptable carriers, vehicles and/or excipients, said packaging having from 7 to 21 dosage units, or multiples thereof, thereby having dosage units for one week of administration or several weeks of administration.

The dosage units are as defined above, i.e. a dosage unit preferably comprises an amount of the ghrelin-like compound or a salt thereof equivalent to from 0.3 μg to 600 mg ghrelin, such as of from 2.0 μg to 200 mg ghrelin, such as from 5.0 μg to 100 mg ghrelin, such as from 10 μg to 50 mg ghrelin, such as from 10 μg to 5 mg ghrelin, such as from 10 μg to 1.0 mg ghrelin.

The medical packaging may be in any suitable form for parenteral, in particular subcutaneous administration. In a preferred embodiment the packaging is in the form of a cartridge, such as a cartridge for an injection pen, the injection pen being such as an injection pen known from insulin treatment.

When the medical packaging comprises more than one dosage unit, it is preferred that the medical packaging is provided with a mechanism to adjust each administration to one dosage unit only.

Preferably, a kit contains instructions indicating the use of the dosage form to achieve a desirable affect and the amount of dosage form to be taken over a specified time period. Accordingly, in one embodiment the medical packaging comprises instructions for administering the pharmaceutical composition.

### Compounds for Nasal Administration

The compounds of the present invention may be formulated for nasal administration. The solutions or suspensions are applied directly to the nasal cavity by conventional means, for example with a dropper, pipette or spray. The compositions may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

### Compounds for Aerosol Administration

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Compositions administered by aerosols may be prepared, for example, as solutions in saline, employing benzyl alcohol or other suitable preservatives, absorption promoters to enhance bioavailability, employing fluorocarbons, and/or employing other solubilizing or dispersing agents.

### Pharmaceutically Acceptable Salts

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

### Dosing Regimes

The pharmaceutical preparations described herein are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. When desired, compositions can be prepared with enteric coatings adapted for sustained or controlled release administration of the active ingredient.

In one aspect of the present invention, a suitable dose of the compositions described herein is administered in pharmaceutically effective amounts to an individual in need of such treatment. Herein, “pharmaceutically effective amounts”, is defined as an administration involving a total amount of each active component of the medicament or pharmaceutical composition or method that is sufficient to show a meaningful patient benefit. The term “unit dosage form” as used herein refers to physically discrete units suitable as unitary dosages for human and animal subjects, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

The dosage requirements will vary with the particular drug composition employed, the route of administration and the particular subject being treated. Ideally, a patient to be treated by the present method will receive a pharmaceutically effective amount of the compound in the maximum tolerated dose, generally no higher than that required before drug resistance develops. Suitable dosing regimens are preferably determined taking into account factors well known in the art including type of subject being dosed; age, weight, sex and medical condition of the subject; the route of administration; the renal and hepatic function of the subject; the desired effect; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug.

It should be noted that the normal ghrelin response which occurs before a meal is a short-lived surge in plasma concentrations of ghrelin and that due to the relative short half life of the peptide an i.v. injection of ghrelin will ensure that a similar short-lived peak on ghrelin concentrations can be obtained. The administration route must ensure that the non-degraded, bioactive form of the peptide will be the dominating form in the circulation, which will reach the ghrelin receptors and stimulate these. Thus, in order to obtain the maximum effect of the medicament it is preferably administered from one to three times daily, each administration being within 90 minutes of a meal, such as within 85 minutes of a meal, such as within 80 minutes of a meal, such as within 75 minutes of a meal, such as within 70 minutes of a meal, such as within 65 minutes of a meal, such as within 60 minutes of a meal, such as within 55 minutes of a meal, such as within 50 minutes of a meal, such as within 45 minutes of a meal, such as within 40 minutes of a meal, such as within 35 minutes of a meal, such as within 30 minutes of a meal, such as within 25 minutes of a meal, such as within 20 minutes of a meal, such as within 15 minutes of a meal, such as within 10 minutes of a meal, such as within 5 minutes of a meal. More preferred the medicament is administered prior to each main meal, such as administered three times daily.

For the present invention the dosage will vary depending on the compound employed and the mode of administration. Dosage levels will vary between about 0.01 μg/kg body weight to 1 g/kg body weight daily, preferably between about 0.01 μg/kg body weight to 1 mg/kg body weight, such as between 0.01 to 10 μg/kg body weight, for example about 0.01 μg/kg body weight. In one preferred embodiment, the dosage level is about 10 μg/kg body weight For all methods of use disclosed herein for the compounds, the daily oral dosage regimen will preferably be from about 0.01 μg to about 80 mg/kg of total body weight. The daily parenteral dosage regimen about 0.01 μg to about 80 mg/kg of total body weight. The daily topical dosage regimen will preferably be from 0.01 μg to 150 mg, administered one to four, preferably two or three times daily. The daily inhalation dosage regimen will preferably be from about 0.01 μg/kg to about 1 mg/kg per day. It will also be recognized by one of skill in the art that the optimal quantity and spacing of individual dosages of a compound or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable salt thereof given per day for a defined number of days, can be ascertained by those skilled in the art using conventional course of treatment determination tests.

Furthermore, since the “effective level” is used as the preferred endpoint for dosing, the actual dose and schedule can vary, depending on interindividual differences in pharmacokinetics, drug distribution, and metabolism. The “effective level” can be defined, for example, as the blood or tissue level desired in the patient that corresponds to a concentration of one or more compounds according to the invention.

In one preferred embodiment, the compounds of the present invention are formulated as described in the literature for an administration route selected from: buccal delivery, sublingual delivery, transdermal delivery, inhalation and needle-free injection, such as using the methods developed by Powderjet.

For inhalation, the compounds of the present invention can be formulated as using methods known to those skilled in the art, for example an aerosol, dry powder or solubilized such as in micro droplets, preferably in a device intended for such delivery (such as commercially available from Aradigm, Alkerme or Nektar).

### Bolus Administration

From a molecular pharmacological point-of-view it is important to note that it has been found that the ghrelin receptor normally is exposed to short-lived surges in the concentrations of the natural agonist ligand, ghrelin. The GHS-R 1a receptor belongs to the class of receptors, so-called G protein coupled receptors or 7™ receptors, that upon continued exposure to an agonist will be desensitised, internalized and down-regulated. These mechanisms, which are inherent to the overall signal transduction system, involve processes such as receptor phosphorylation (which in itself decreases the affinity of the receptor for the agonist) binding of inhibitory proteins such as arrestin (which sterically block the binding of signal transduction molecules such as G proteins). Another part of the agonist mediated desensitization process is receptor internalization (i.e. physical removal of the receptor from the cell surface where it could bind the agonist) as well as receptor down regulation (i.e. decreased production/expression of the receptor). Receptor internalization could after short-lived exposure of the receptor to agonist be followed by a re-sensitization process, where the receptor is dephosphorylated and recycled to the cell surface to be used again. Without being bound by theory, it is believed that, upon prolonged stimulation, which would occur for example during a long-lasting continuous infusion of the agonist, the receptor down-regulation process ensures that the target cell is adjusted in its signal transduction system etc. to this situation.

Accordingly, the present invention relates in one aspect to administration of a secretagogue, such as a ghrelin-like compound, in boluses.

In one preferred embodiment of the present invention, a secretagogue such as ghrelin or a ghrelin-like compound is administered as a bolus in an amount equivalent to 10 μg per kg body weight.

### Methods for Production of Ghrelin

Secretagogue compounds can be produced using techniques well known in the art. For example, a polypeptide region of a secretagogue can be chemically or biochemically synthesized and modified. Techniques for chemical synthesis of polypeptides are well known in the art. (See e.g., Vincent in Peptide and Protein Drug Delivery, New York, N.Y., Dekker, 1990.) Examples of techniques for biochemical synthesis involving the introdction of a nucleic acid into a cell and expression of nucleic acids are provided in Ausubel, Current Protocols in Molecular Biology, John Wiley, 1987-1998, and Sambrook et al., in Molecular Cloning, A Laboratory Manual, 2 d Edition, Cold Spring Harbor Laboratory Press, 1989.

Pharmaceutical compositions containing a compound of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications.

One suitable method for synthetic production of the secretagogue for use in the present invention is described in Example 2 of WO2005014032 (Gastrotech Pharma A/S).

### EXAMPLE

The following example illustrates the invention without limiting it thereto.

### Example I

### Ghrelin in the Treatment of Hyperthyroidism

### Purpose:

1) Normalize the plasma ghrelin level in order to stabilize and reverse the catabolic conditions observed during hyperthyroidism.
2) Normalization of plasma ghrelin may additionally prevent a compensatory up-regulation of ghrelin receptors on the hypothalamic neurons which is usually observed in relation to decreased plasma levels of hormones. It is hypothesized that an up-regulation of the expression of hypothalamic ghrelin receptors contribute to the increased appetite and obesity frequently observed in patients following hyperthyroid diseases.
3) Patients suffering from Grave's disease may benefit from ghrelin also due to the inhibitory effect on the immune system.

### Method:

Two different models of hyperthyroid conditions are established:
1) Hyperthyroid condition without concomitant autoimmune dysfunction imitated by intraperitoneal application of L-thyroxine over 15 days.
2) Autoimmune hyperthyroid condition induced by administration of adenovirus expressing human (Thyroid Stimulating Hormone) TSH receptor.

Administration of L-thyroixine: In 6-wk-old BALB/c mice L-thyroxine is administrated intraperitoneally in doses of 40 mg/kg daily for 15 day.

Adenovirus expressing TSH-R: TSH-R is cloned into an expression vector for adenovirus pAdHM4 which is linealized with PacI and transfected into 293 human embryonal kidney cells with SuperFect (Qiagen) according to the manufacturer's instructions. Recombinant adenovirus expressing TSH-R will then be plaque-purified. Adenovirus is propagated in 293 human embryonal kidney cells and purified through two rounds of CsCl density gradient centrifugation. The viral particle concentration is determined by measuring the absorbance at 260 nm following the incubation of the virus solution in 10 mM Tris-HCl, 1 mM EDTA, and 0.1% SDS at 56° C. for 10 min; an absorbance of 1 corresponds to 1.1×1012 particles/ml. BALB/c 6-wk-old mice are immunized with adenovirus, mice are i.m. injected with 50 μl PBS containing 1×1011 particles of adenovirus expressing TSH receptor or a control virus. The same immunization schedule is repeated twice at 3-wk intervals. Both control mice and the two different mice models for hyperthyroidism are treated with ghrelin (100 μg/kg) or saline s.c. administrated once daily for two weeks. Treatment is initiated after 15 days of L-thyroxine administration or after the second administration of adenovirus administration.

After two weeks of ghrelin administration the mice are sacrificed and thorax blood is collected and hypothalamus and the thyroid gland are dissected.

### Analyses:

Blood samples: TSH (is usually decreased as a negative feedback response to the high level of thyroid hormones). T4 and free T3

TSH antibody titer (measured to evaluate the autoimmune responds in Grave's disease)

Hypothalaums: Quantitative RT-PCR is performed for important appetite regulating peptides like NPY, POMC, GHS-R1a,

Thyroid gland: Determination of the follicular content of thyroiglobulin as measured by quantitative RT-PCR.

### Conclusion:

If the expression level of GHS-R1a in hypothalamus is increased as a response to the decreased level of plasma ghrelin it may contribute to the observed obesity following an episode of hyperthyroid. It has previously been shown that increase in the expression of the ghrelin receptor only by 30% induces a strong increase in food intake and obesity (BMI>30) as shown by a family with a single point mutation in the ghrelin receptor gene promoter (ref Hingstrup L and Pedersen O). It is assumed that ghrelin administration, if it prevent an increase in expression level of the hypothalamic receptor expression, also prevent the compensatory increase in food intake.

1-33. (canceled)

34. A method for prophylaxis or treatment of ghrelin deficiency in an individual in need thereof comprising administering an effective dose of a growth hormone secretagogue.

35. The method according to claim 34, wherein said ghrelin deficiency can be diagnosed in a patient using the method for measuring ghrelin levels described by Cummings et al in the New England Journal of Medicine, 2002, 346(21):1623-30, wherein ghrelin deficiency can be diagnosed when an individual is measured as having a ghrelin level lower than 175 fmol/ml at breakfast peak.

36. The method according to claim 34, wherein said ghrelin deficiency is caused by a pathological condition.

37. The method according to claim 34, wherein said ghrelin deficiency is caused by a medical treatment.

38. The method according to claim 34, wherein said individual in need is suffering from hyperthyroidism.

39. The method according to claim 38, wherein said hyperthyroidism is caused by one or more of the following: Grave's disease, drugs containing a high level of iodine, thyroiditis, subacute thyroiditis, postpartum thyroiditis, loss of feedback control of thyroid hormone producing cells, toxic nodular goiter, excessive doses of thyroid hormone or thyroid medication.

40. The method according to claim 34, wherein said individual in need is suffering from or at risk of suffering from-fully or partially disrupted epithelium in the GI tract.

41. The method according to claim 40, wherein said disruption of the epithelium is caused by one or more of: a pathological condition, a genetic disease, or a medical treatment.

42. The method according to claim 41, wherein said medical treatment is selected from chemotherapy and/or radiotherapy.

43. The method according to claim 41, wherein said pathological condition is Gastristis.

44. The method according to claim 34, wherein said individual in need is suffering from, or at risk of suffering from, a pathological condition associated with insulin resistance.

45. The method according to claim 44, wherein said individual in need has hyperinsulinemia.

46. The method according to claim 44, wherein said pathological condition is selected from the group consisting of: polycystitic ovary syndrome, acromegaly, primary/secondary hypogonadism, Non-AlcoholicFatty Liver Disease (NAFLD) and Type I Diabetes Mellitus.

47. The method according to claim 34, wherein the individual in need, has been, will be, or is currently treated using chemotherapy, comprising administering an effective dose of a growth hormone secretagogue.

48. A method for preventing weight increase in an individual either
a) being converted from a hyperthyroidic state to euthyroid state or
b) in remission from being converted from a hyperthyroidic state to euthyroid state
comprising administering an effective dose of a growth hormone secretagogue.

49. The method according to claim 48, wherein the individual is suffering from, or in remission from suffering from, Grave's disease.

50. The method according to claim 48, wherein the individual is suffering from, or in remission from suffering from, thyroiditis.

51. The method according to claim 48, wherein the thyroiditis is subacute thyroiditis or postpartum thyroiditis.

52. The method according to claim 48, wherein the thyroiditis is De Quervain thyroiditis.

53. The method according to claim 48, wherein the individual is suffering from, or in remission from suffering from, solitary adenoma.

54. The method according to claim 48, wherein the individual is suffering from, or in remission from suffering from, toxic nodular goitre.

55. The method according to claim 48, wherein the individual is suffering from, or in remission from suffering from, symptoms caused by an excessive dose of thyroid hormone, for instance an individual who has taken a form of thyroid medication that contains T3.

56. The method according to claim 48, wherein the individual is suffering from, or in remission from suffering from, symptoms caused by loss of feedback control of thyroid producing cells.

57. The method according to claim 48, wherein the growth hormone secretagogue is given in combination with a medicament for treatment of hyperthyroidism.

58. The method according to claim 57, wherein the medicament for treatment of hyperthyroidism is one or more of:
A) antithyroid drugs and/or
B) surgery and/or
C) radioiodine;
optionally in combination with drugs targeting the cardiovascular system.

59. The method according to claim 34, wherein the growth hormone secretagogue is ghrelin or a pharmaceutically acceptable salt thereof.

60. The method according to claim 34, wherein the growth hormone secretagogue is a ghrelin-like compound or a pharmaceutically acceptable salt thereof
wherein the ghrelin-like compound comprises a structure defined by formula I
Z¹-(X¹)—(X²)—(X³)ₙ-Z², wherein
Z¹ is an optionally present protecting group
each X¹ is independently selected from an amino acid, wherein said amino acid is selected from naturally occurring and synthetic amino acids,
X² is any amino acid selected from naturally occurring and synthetic occurring amino acids, said amino acid being modified with a bulky hydrophobic group, preferably an acyl group, or a fatty acid,
each X³ is independently selected from an amino acid, wherein said amino acid is selected from naturally occurring and synthetic amino acids,
wherein one or more of X¹ and X³ optionally may be modified by a bulky hydrophobic group, preferably an acyl group, or a fatty acid,
Z² is an optionally present protecting group,
m is an integer in the range of from 1-10
n is 0 or an integer in the range of from 1-35.

61. The method according to claim 34, wherein the growth hormone secretagogue is in a formulation for oral, nasal, pulmonal, parenteral, intravenous, intramuscular or subcutaneous administration.

62. The method according to claim 34, wherein the growth hormone secretagogue is a ghrelin-like compound, or a pharmaceutically acceptable salt thereof.

63. The method according to claim 34, wherein the growth hormone secretagogue is comprised by a formulation, wherein said growth hormone secretagogue is a lyophilisate and the formulation further comprises a solvent, said lyophilisate and said solvent being in separate compartments until administration.

64. The method according to claim 34, wherein the growth hormone secretagogue, is comprised by a solution of the growth hormone secretagogue.

65. The method according to claim 34, wherein the growth hormone secretagogue is administered in a concentration equivalent to from 10 ng to 10 g ghrelin per kg bodyweight.

66. The method according to claim 34, wherein the growth hormone secretagogue is administered as a bolus prior to or during a meal, said bolus comprising an amount of the ghrelin-like compound or a salt thereof equivalent to from 0.3 μg to 600 mg ghrelin.

67. The method according to claim 48, wherein the growth hormone secretagogue is ghrelin or a pharmaceutically acceptable salt thereof.

68. The method according to claim 48, wherein the growth hormone secretagogue is a ghrelin-like compound or a pharmaceutically acceptable salt thereof
wherein the ghrelin-like compound comprises a structure defined by formula I
Z¹-(X¹)ₘ—(X²)—(X³)ₙ-Z², wherein
Z¹ is an optionally present protecting group
each X¹ is independently selected from an amino acid, wherein said amino acid is selected from naturally occurring and synthetic amino acids,
X² is any amino acid selected from naturally occurring and synthetic occurring amino acids, said amino acid being modified with a bulky hydrophobic group, preferably an acyl group, or a fatty acid,
each X³ is independently selected from an amino acid, wherein said amino acid is selected from naturally occurring and synthetic amino acids,
wherein one or more of X¹ and X³ optionally may be modified by a bulky hydrophobic group, preferably an acyl group, or a fatty acid,
Z² is an optionally present protecting group,
m is an integer in the range of from 1-10
n is 0 or an integer in the range of from 1-35.

69. The method according to claim 48, wherein the growth hormone secretagogue is in a formulation for oral, nasal, pulmonal, parenteral, intravenous, intramuscular or subcutaneous administration.

70. The method according to claim 48, wherein the growth hormone secretagogue is a ghrelin-like compound, or a pharmaceutically acceptable salt thereof.

71. The method according to claim 48, wherein the growth hormone secretagogue is comprised by a formulation, wherein said growth hormone secretagogue is a lyophilisate and the formulation further comprises a solvent, said lyophilisate and said solvent being in separate compartments until administration.

72. The method according to claim 48, wherein the growth hormone secretagogue, is comprised by a solution of the growth hormone secretagogue.

73. The method according to claim 48, wherein the growth hormone secretagogue is administered in a concentration equivalent to from 10 ng to 10 g ghrelin per kg bodyweight.

74. The method according to claim 48, wherein the growth hormone secretagogue is administered as a bolus prior to or during a meal, said bolus comprising an amount of the ghrelin-like compound or a salt thereof equivalent to from 0.3 μg to 600 mg ghrelin.
